(19) United States
(12) Patent Application Publication
(10) Pub. No.: US 2016/0346466 A1
(43) Pub. Date: Dec. 1, 2016

(54) MICROFLUIDIC DEVICE

(71) Applicant: National University of Singapore, Singapore (SG)

(72) Inventors: Hao Wang, Singapore (SG); Zhuolin Xiang, Singapore (SG); Chengkuo Lee, Singapore (SG)

(73) Assignee: National University of Singapore, Singapore (SG)

(21) Appl. No.: 15/163,677

(22) Filed: May 25, 2016

(30) Foreign Application Priority Data
May 25, 2015 (SG) SG 10201504101S

(51) Int. Cl. A61M 5/142; A61M 5/172; A61M 37/00; A61M 5/168; A61M 39/24

(52) U.S. Cl. CPC A61M 5/14248; A61M 5/16881; A61M 39/24; A61M 37/0015; A61M 5/172; A61M 5/1723; F16K 2099/0086

(57) ABSTRACT

A microfluidic device (100) is disclosed. The device comprises a triboelectric sensor (102); an elastically deformable pump (104) arranged to transfer fluid to at least one fluid outlet (108) and triboelectrically activate the sensor; and first and second check valves (106a, 106b) respectively arranged at the inlet and outlet of the pump to control fluid transfer in and out of the pump. When the pump is actuated in conjunction with the check valves to transfer a volume of fluid, at least a portion of the sensor is triboelectrically activated according to an amount of deformation of the pump to generate a corresponding output voltage signal for enabling the volume of fluid transferred by the deformed pump to be determined. A corresponding method of using the device is also disclosed.

### FIELD

The present invention relates to a microfluidic device.

### BACKGROUND

Wearable medical devices based on lab-on-chip (LoC) technology have received major attention recently owing to their considerable practicability for health monitoring and disease treatment. The interest, within the medical community, for wearable medical monitoring systems arises from a need to monitor patients from a distance and over long periods of time. Such on-body monitoring devices may alert the patients of any imminent health hazard, and hence facilitate rapid corrective clinical action outside of the hospital environment. Conventionally, wearable sensors for health monitoring have been explored, including devices that measure hydration, strain, glycaemia, metabolic acid, and cardiorespiratory signals. While these wearable sensors monitor important health parameters, there is still a strong need for wearable systems designed for chronic diseases treatment such as hypertension, osteoporosis and diabetes. Preferably in such cases, patients should have access to convenient means for self-administering transdermal drug delivery. To this end, several kinds of skin patches (e.g. micro-needle skin patch for insulin delivery, and pain patch to relief pain) have been developed and demonstrated to satisfy those said requirements. With drug(s) coated on a side of the skin patches, they are suitable for self-administrated drug delivery. Nonetheless, the drug coating method using skin patches may only administer limited drug volume, and also the specific dosage to be delivered cannot be controlled.

However, for treating diseases such as type-2 diabetes and osteoporosis, multiple drug injections per day with dosage control of each injection are necessary; the aforementioned skin patches are thus unable to fully meet these requirements. Thus, a mechanism that is able to precisely provide a large delivery volume is desirable for a wearable skin patch drug delivery system, but unfortunately yet to be developed. Moreover, a standalone wearable drug delivery skin patch with capability for health monitoring, signal processing, interfacing with external cloud computing apparatus, and having an built-in energy source to power components (e.g. integrated circuits (ICs), a microprocessor, liquid-crystal display (LCD) reading panel, drug delivery and control actuators, and diversified sensors such as a glucose sensor) are desirable.

Another separate issue is that traditional drug delivery method using hypodermic needles tends to be an unpleasant and stressful experience for many patients. So, micro-needle-based transdermal drug delivery approaches have been investigated in the art by changing different kinds of materials and configurations of the micro-needle(s). Presently, for clinically relevant applications, a main concern of using conventional micro-needles is the safety issues posed by needle breakage after skin penetration (by the micro-needles). High aspect ratio and sharp tips made of rigid materials are often necessary properties of conventional micro-needles to provide successful and reliable skin penetration. Normally, micro-needles formed from materials (e.g. nickel, stainless steel, and silicon) with high Young's modulus may avoid such needle breakage. However, those materials lack biocompatibility, which is a key requirement for such medical devices. On the other hand, micro-needles made of biocompatible polymers or natural fibers then suffer from having low mechanical strength. Typically, the consequential needle breakage is caused by very high buckling force attributed to the deforming of skin surface during skin penetration, or by any lateral movement collectively experienced by the micro-needles and skin surface during drug administration.

One object of the present invention is therefore to address at least one of the problems of the prior art and/or to provide a choice that is useful in the art.

### SUMMARY

According to a 1^{st} aspect, there is provided a microfluidic device comprising: a triboelectric sensor; an elastically deformable pump arranged to transfer fluid to at least one fluid outlet and triboelectrically activate the sensor; and first and second check valves respectively arranged at the inlet and outlet of the pump to control fluid transfer in and out of the pump, wherein when the pump is actuated in conjunction with the check valves to transfer a volume of fluid, at least a portion of the sensor is triboelectrically activated according to an amount of deformation of the pump to generate a corresponding output voltage signal for enabling the volume of fluid transferred by the deformed pump to be determined.

Advantageously, the device enables the volume of fluid delivered by the pump to be precisely determined using the triboelectric sensor, which is concurrently activated when the pump is manually pressed to make the fluid delivery. Such as, the delivery volume can beneficially be monitored, which is crucial in certain medical applications (in which the device is adaptable for usage) such as for insulin delivery, where the delivery dosage needs to be accurately controlled and measured. Moreover, the device is a pure passive device, and has a small form factor.

Preferably, the pump may be arranged to abut the sensor.

Preferably, the device may further comprise at least one fluid reservoir for holding a fluid drug medication, which is to be delivered together with the volume of fluid.

Preferably, the fluid drug medication may include insulin.

Preferably, the pump may include being formed of polydimethylsiloxane (PDMS).

Preferably, the device may further include a plurality of triboelectric energy harvesters configured in a stacked arrangement for generating a collective output voltage signal.

Preferably, the device may further comprise a convertor circuit, and at least one power component, wherein the convertor circuit is configured to convert the collective output voltage signal into electricity for powering the power component.

Specifically, the power component may include an integrated circuit, a microprocessor, and a LCD reading panel.

Preferably, the sensor may include first and second portions in opposing arrangement and separated by an air gap, the first portion having at least a first dielectric layer, and the second portion having at least a second dielectric layer coated with a layer of parylene.

Preferably, the first and second dielectric layers may be formed of polydimethylsiloxane (PDMS).

Preferably, a surface of the first electric layer may be further coated with a metal layer, in which the surface is in opposition to the second portion.

Preferably, the metal layer may include an aluminium layer.

Preferably, a surface of the second electric layer may also further be regularly arranged with a plurality of micro-features, in which the surface is in opposition to the first portion.

Preferably, each micro-feature may have a pyramid shape.

Preferably, the second portion may be arranged adjacent to the pump.

Preferably, the device may further comprise a micro-needle array device which includes: a flexible substrate formed with a plurality of base protrusions which are elastically deformable, each protrusion having a plurality of recesses for enabling fluid transfer; and a plurality of micro-needles co-axially arranged on the respective protrusions, the micro-needles being substantially rigid. When a lateral force is applied onto the device, the micro-needles are displaced off-axis relative to the protrusions due to deformation of the protrusions, and when the lateral force is removed, the micro-needles return to the co-axial arrangement; and wherein the device is incorporated with the micro-needle array and a plurality of dry adhesive patches to form a skin patch adapted for transdermal drug delivery.

Preferably, the flexible substrate may be formed of polydimethylsiloxane (PDMS), and the micro-needles may be formed of SU-8 photoresist or maltose.

According to a 2^{nd} aspect, there is provided a method of using a microfluidic device, which includes a triboelectric sensor; an elastically deformable pump arranged to transfer fluid to at least one fluid outlet and triboelectrically activate the sensor; and first and second check valves respectively arranged at the inlet and outlet of the pump to control fluid transfer in and out of the pump. The method comprises: actuating the pump in conjunction with the check valves to transfer a volume of fluid, and to cause at least a portion of the sensor to be triboelectrically activated according to an amount of deformation of the pump; and generating a corresponding output voltage signal by the activated sensor for enabling the volume of fluid transferred by the deformed pump to be determined.

According to a 3^{rd} aspect, there is provided a micro-needle array device comprising: a flexible substrate formed with a plurality of base protrusions which are elastically deformable, each protrusion having a plurality of recesses for enabling fluid transfer; and a plurality of micro-needles co-axially arranged on the respective protrusions, the micro-needles being substantially rigid, wherein when a lateral force is applied to the device, the micro-needles are displaced off-axis relative to the protrusions due to elastic deformation of the protrusions caused by the force, and when the force is removed, the micro-needles return to the co-axial arrangement.

Advantageously, the micro-needles are configured to tolerate deformation associated with skin stretching (due to relative movement from user handling), but without suffering from needle breakage, when applied onto a user for associated medical treatment(s).

Preferably, the flexible substrate may be formed of polydimethylsiloxane (PDMS), and the micro-needles may be formed of SU-8 photoresist or maltose.

It should be apparent that features relating to one aspect of the invention may also be applicable to the other aspects of the invention.

These and other aspects of the invention will be apparent from and elucidated with reference to the embodiments described hereinafter.

### BRIEF DESCRIPTION OF THE DRAWINGS

Embodiments of the invention are disclosed hereinafter with reference to the accompanying drawings, in which:

FIG. 1A is a cross-sectional view of a micro-fluidic device, which includes a triboelectric sensor, an elastically deformable pump, and first and second check valves, according to a first embodiment;

FIG. 1B depicts sequences relating to activation of the sensor of the device shown in FIG. 1A and a magnified view of a portion of the sensor shown in FIG. 1A, showing a parylene layer used to encapsulate the sensor, as well as being coated on the bottom of the pump of the device shown in FIG. 1A;

FIG. 2 shows an exemplary operation of the pump (when actuated), in conjunction with the first and second check valves of the device shown in FIG. 1A;

FIG. 3 shows a cross-sectional view and a perspective view of each check valve of the device shown in FIG. 1A;

FIG. 4 is a flow diagram of a method of using the device shown in FIG. 1A;

FIG. 5 is a tabulation table showing details of different groups of samples for optimizing PDMS thickness and triboelectric contact surfaces of the sensor shown in FIG. 1A;

FIGS. 6A-6C depict evaluation results relating to optimization and characterization of the triboelectric contact surfaces of the sensor shown in FIG. 1A;

FIG. 7A show a photographical sequence demonstrating drug delivery with fluid volume sensor monitoring using the device shown in FIG. 1A;

FIG. 7B shows evaluation results related to optimization for the air gap separating first and second portions of the sensor shown in FIG. 1A;

FIG. 7C shows evaluation results related to delivery volume calibration of the device shown in FIG. 1A, via using different measuring methods;

FIG. 8 shows a perspective view of a lab-on-chip (LoC) drug delivery skin patch, which incorporates the device shown in FIG. 1A, a micro-needle array device, and a plurality of dry adhesive patches;

FIGS. 9A-9C show steps for fabricating the skin patch shown in FIG. 8;

FIG. 10A shows an optical image of a micro-needle array device (at a scale of 300 μm), wherein the micro-needles are formed of SU-8 photoresist, according to a second embodiment, and a magnified view of an individual micro-needle of the array device shown in FIG. 10A (at a scale of 150 μm);

FIG. 10B shows resilient bending of the micro-needles of the array device shown in FIG. 10A (without breakage), when a lateral force is applied onto the device and then removed;

FIG. 10C are photographs demonstrating resilient bending of a micro-needle of the array device shown in FIG. 10A, when relative movement between skin and the micro-needle occurs after skin penetration;

FIG. 10D show an optical image of a micro-needle array device (at a scale of 300 μm), wherein the micro-needles are formed of maltose, according to a third embodiment, and a magnified view of an individual micro-needle of the array device shown in FIG. 10D (at a scale of 150 μm);

FIG. 10E shows resilient bending of the micro-needles of the array device shown in FIG. 10D (without breakage), when a lateral force is applied onto the device and then removed;

FIGS. 11A-11E illustrate evaluation results and images related to optimizing PDMS stiffness and pillar angular of the micro-needles shown in FIGS. 10A and 10D for withstanding higher buckling force and improving success rate of skin penetration;

FIG. 12 is a tabulation table showing success rate of skin penetration for micro-needles with SU-8 or maltose sharp tips, when the mix ratio of PDMS is changed from 1:4 to 1:10;

FIGS. 13A-13B show usage of double drawing lithography for manufacturing the micro-needle array device shown in FIG. 10A or 10D; and

FIGS. 14A-14B illustrate evaluation results and images related to using the skin patch shown in FIG. 8 for monitoring and controlling a dosage for insulin delivery.

### DETAILED DESCRIPTION OF PREFERRED EMBODIMENTS

### 1. Experiment

### 1.1 Design Structure and Working Principle of a Triboelectric Sensor

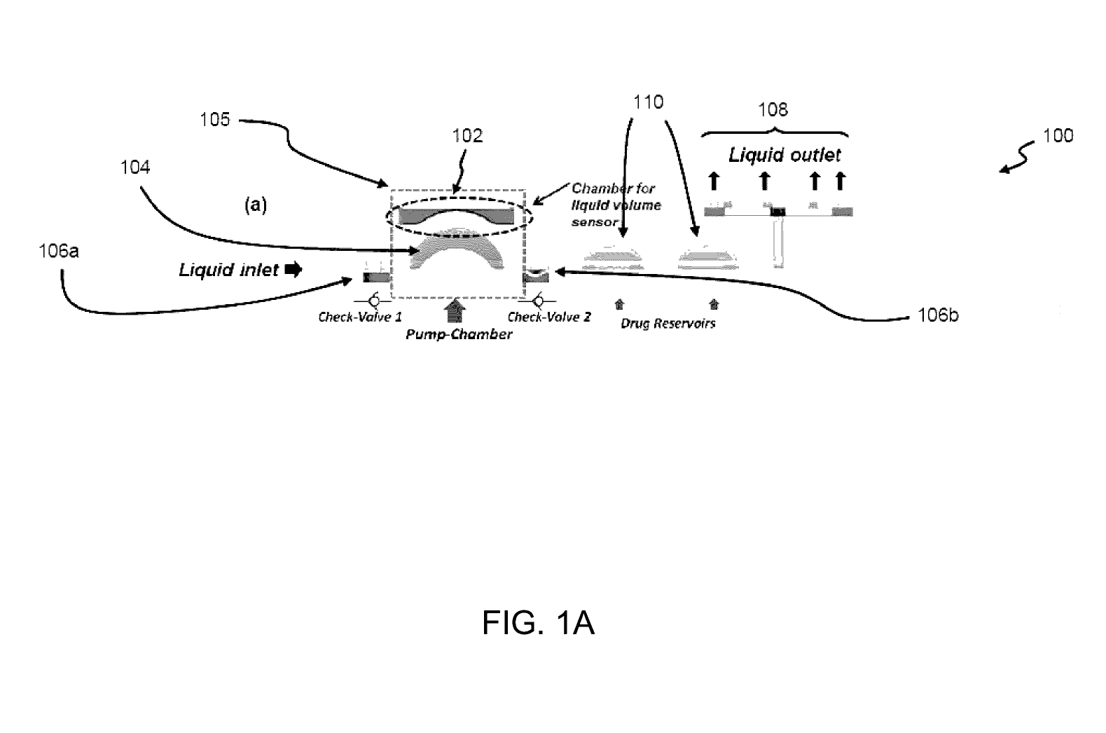

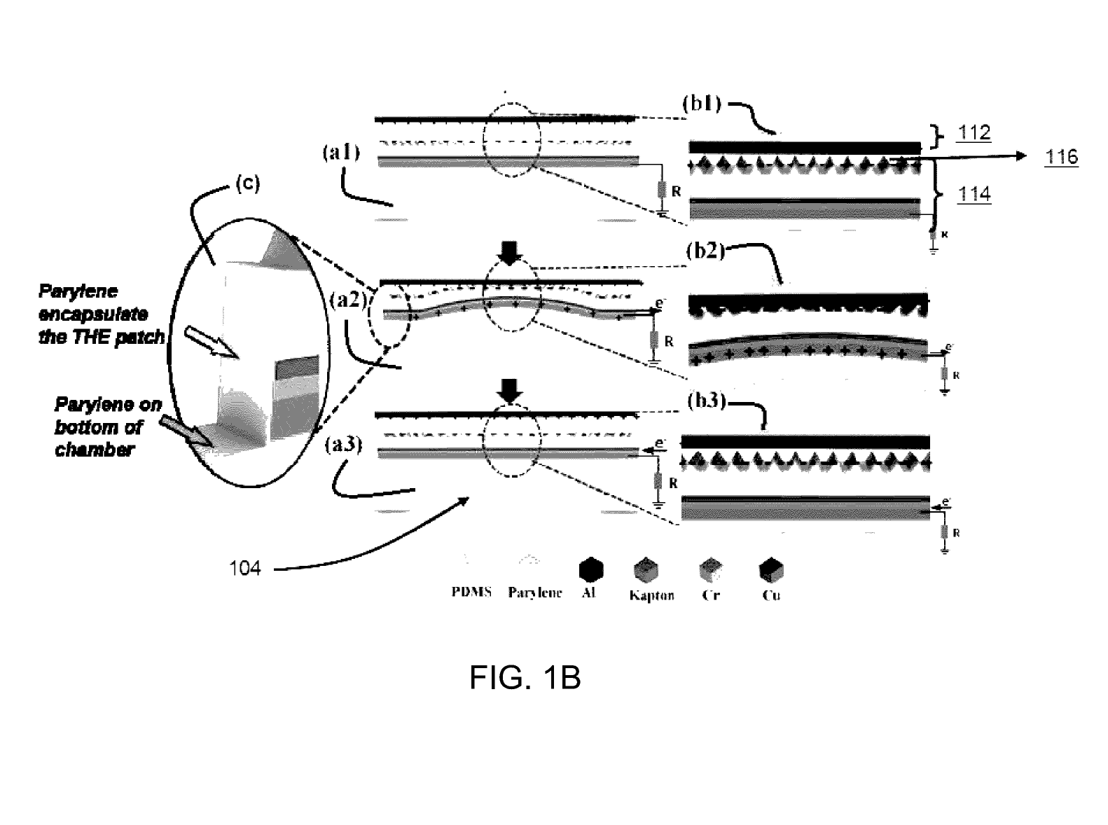

A microfluidic device 100 (hereafter abbreviated as the first device 100), which includes a triboelectric sensor 102, an elastically deformable pump 104 (which is polymer-based), and first and second check valves 106a, 106b, is disclosed in FIG. 1A, based on a first embodiment. It is to be appreciated that the sensor 102 may also be known as a triboelectric fluid delivery volume sensor/fluid flow sensor, since the sensor 102 is adapted to measure an amount of fluid displaced by the pump 104 when defomably actuated (e.g. manually pressed using a finger). Also, the pump 104 and the first and second check valves 106a, 106b together form a microfluidic control system. The pump 104 is arranged to transfer fluid (e.g. liquid or air) to at least one fluid outlet 108, and in conjunction, triboelectrically activate the sensor 102, when being deformably actuated. In this embodiment, liquid is used as an example of fluid, but is not to be construed as limiting. Also, the sensor 102 and pump 104 together define a pump-chamber 105 of the first device 100. Then, the first and second check valves 106a, 106b are respectively arranged at the inlet and outlet of the pump 104 to control fluid transfer in and out of the pump 104. That is, the first and second check valves 106a, 106b are arranged in series, with the pump 104 arranged intermediate the two check valves 106a, 106b. Specifically, when the pump 104 is actuated in conjunction with the two check valves 106a, 106b to transfer a volume of fluid (e.g. to be dispensed via the fluid outlets 108 of the device 100), at least a portion of the sensor 102 is triboelectrically activated based on an amount of deformation of the pump 104 to generate a corresponding output voltage signal for enabling the volume of fluid transferred by the deformed pump 104 to be determined.

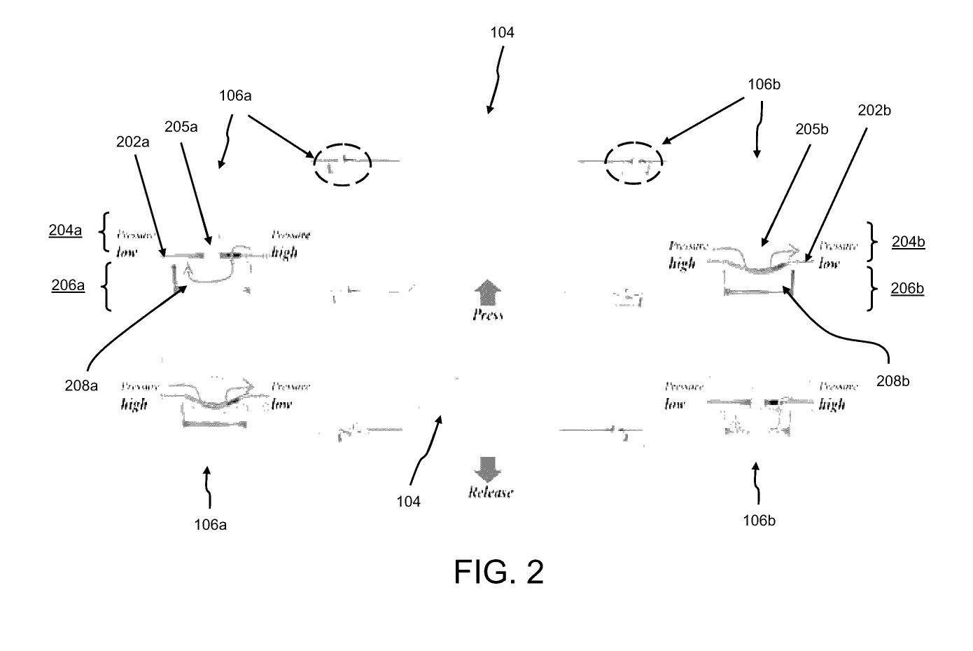

It is to be appreciated that the first device 100 may find application as a lab-on-chip (LoC) drug delivery skin patch with manually-controlled drug delivery and dosage volume monitoring (i.e. see Section 2.3), but is only one of the many applications possible. Hence, as depicted in FIG. 1A, the first device 100 may optionally include several fluid/drug reservoirs 110 in series arrangement with the second check valve 106b for holding fluid drug medications (e.g. insulin or the like), which are to be transferred together with the volume of fluid when the pump 104 is actuated. Needless to say, the first device 100 includes necessary microfluidic channels to interconnect the pump 104, the first and second check valves 106a, 106b, the fluid reservoirs 110, and the fluid outlets 108 in fluid communication. By deforming the pump 104 (e.g. manually pressing the pump 104), fluid is sucked into an associated microfluidic channel linked to the inlet of the first device 100 and then delivered out of the fluid outlets 108, as shown in FIG. 1A. The detailed structure and working principle of the pump 104 and two check valves 106a, 106b are to be elaborated later below, with reference to FIG. 2.

A triboelectric energy harvester (TEH), which forms the main component of the sensor 102, is integrated directly above and adjacent to the pump-chamber 105. That is, the sensor 102 is primarily made from a TEH. It is to be appreciated that the sensor 102 (configured to use the triboelectric sensing mechanism) is purposefully devised, and the sensing mechanism has conventionally been investigated for use in chemical sensors, pressure sensors, motion sensors, and tactile sensors, because it is easy to fabricate devices to make use of the sensing mechanism, which also advantageously provides a self-powering feature. Here a triboelectric layer pair with the same area as the pump-chamber 105 is assembled, e.g. having an area of 1.2×1.2 cm² for sensing liquid volume. The working principle and detailed structure of the fabricated TEH of the sensor 102 is depicted in FIG. 1B.

Specifically, the TEH includes first and second portions 112, 114 in opposing arrangement and separated by an air gap 116 (i.e. a spacing), wherein the first portion 112 has at least a first dielectric layer, and the second portion 114 has at least a second dielectric layer coated with a layer of parylene. It is to be appreciated that the first and second portions 112, 114 together form the triboelectric layer pair. Also, the second portion 114 is arranged adjacent to the pump 104 in this instance. Both the first and second dielectric layers are formed of polydimethylsiloxane (PDMS), but is not to be construed as limiting. A surface of the second electric layer is regularly arranged with a plurality of micro-features/patterns (e.g. each as a pyramid shape), in which the defined surface is in opposition to the first portion 112. The plurality of micro-features/patterns is beneficially used to enhance the contact surface area and an amount of output voltage generated by the TEH. Immediately underneath the second dielectric layer, a layer of Kapton tape is attached as an intermediate layer to facilitate depositing a layer of metal (e.g. a Copper (Cu)) to act as an electrode. To have better adhesion between the Cu layer and the Kapton layer, a Chromium (Cr) layer is deposited before forming the Cu layer. In order to protect the Cu layer, a parylene layer is also coated by CVD onto the whole surface of the TEH, including the second dielectric layer (as already mentioned). Then the TEH is assembled in a PDMS chamber with an Aluminium (Al) layer coated on a surface of the first electric layer, in which the defined surface is in opposition to the second portion 114. It is also to be appreciated that other suitable metals, besides Al, may be used as well.

Now referring to FIG. 1B (which 1B shows sequences for activating the sensor 102 by pressing the pump 104), in an original position where the sensor 102 is not yet activated, the first and second portions 112, 114 are not yet in contact, being separated by the air gap 116 (i.e. see drawings labelled as (a1) and (b1) in FIG. 1B). When the bottom layer of the pump 104 is pressed (i.e. deformably actuated), the fluid in the pump 104 is compressed and the top layer of the pump 104 is also deformed as a consequence. The top layer of the pump 104 then consequently abuts the second portion 114 of the sensor 102, and deforming the second portion 114 in the process. The deformation induces a direct contact between the second portion 114 and the first portion 112 of the TEH, resulting in charge transport between them (i.e. see drawings labelled as (a2) and (b2) in FIG. 1B). According to the triboelectric theory, electrons are transferred from the parylene to the Al layer during the electrification process, since parylene is triboelectrically negative and Al is triboelectrically positive. The change of the negative charges on the surface of the parylene induces positive charges on the Cu layer, in turn driving free electrons to flow from the Cu layer to the ground, resulting in a first output voltage signal being generated. Once the pressing is released from the pump 104, the parylene and Al layers are separated (i.e. see drawings labelled as (a3) and (b3) in FIG. 1B). The recovery of the surface negative charge on parylene layer induces a backflow of electrons from ground to the Cu layer. A second output voltage signal is thus generated, which flows in opposite direction to the first output voltage signal. It is to be appreciated that the first output voltage signal is generated by the contact between parylene and Al layers, and the magnitude of the signal generated is determined by the actual contact area. In connection, this said contact area is determined by an amount of deformation suffered by the pump 104 (from the pressing), which is then related with the volume of fluid transferred/delivered by the actuated pump 104. Thus, based on the first output voltage signal, the transferred volume can be measured and determined.

To avoid any potential interference from the backside of the TEH, a surface of the pump 104, which is in contact with the second portion 114 of the TEH, is also coated with parylene as shown in the magnified view labelled as (c) in FIG. 1B. Thus, the backside of the TEH and the said surface of the pump 104 are formed of a same material, and contact between these two interfaces will not generate any unintended output voltage.

To generate the highest voltage possible by the TEH, the surface materials of the triboelectric layer pair are to be optimized. Moreover, the air gap 116 separating the first and second portions 112, 114 of the sensor 102 is critical to ensure an accuracy of fluid volume measurement by the sensor 102. So, to have a good measurement accuracy of the proposed sensor 102, the air gap 116 is also to be optimized.

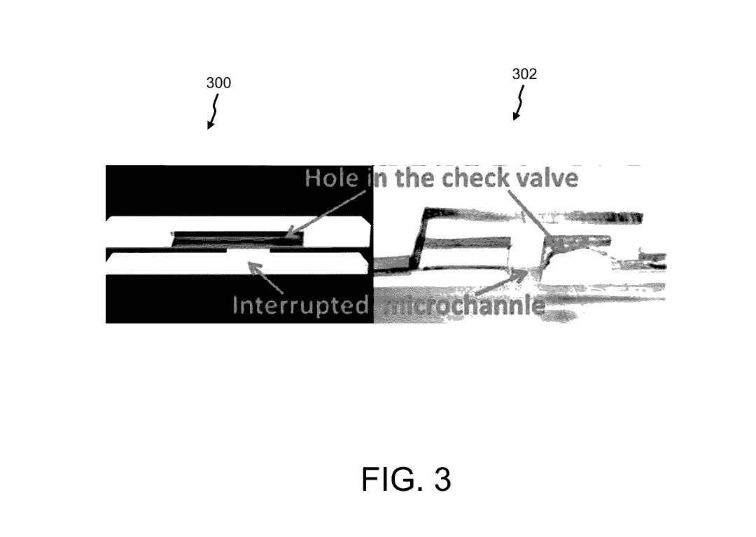

FIG. 2 shows the working principle and operation of the pump 104, in conjunction with the first and second check valves 106a, 106b. Each check valve 106a, 106b comprises three parts: a (fairly thin) PDMS membrane layer 202a, 202b formed with an associated hole, a top layer 204a, 204b (together with a bottom layer 206a, 206b) which defines a fluid channel that can reversibly be interrupted by a protruding portion 205a, 205b (to form a discontinuous channel), and the bottom layer 206a, 206b that has a fluid chamber 208. The membrane layer 202a, 202b is arranged intermediate the top and bottom layers 204a, 204b, 206a, 206b. Each check valve 106a, 106b is formed by bonding together the aforementioned three layers 202a, 202b, 204a, 204b, 206a, 206b. In conjunction, FIG. 3 shows a cross-sectional view 300 and a perspective view 302 of each check valve 106a, 106b. For the first check valve 106a, the hole in the associated membrane layer 202a, 202b is arranged to be in direct fluid communication with the pump 104 at all times, whereas for the second check valve 106b, fluid communication of the pump 104 with the hole of the associated membrane layer 202a, 202b (of the second check valve 106b) is however blocked by the associated protruding portion 205b, until the said membrane layer is deformed by fluid pressure to grant access.

It is to be appreciated that only the contact area between the protruding portion 205a, 205b of the top layer 204a, 204b and membrane layer 202a, 202b is not to be treated with Oxygen plasma, but other portions of the protruding portion 205a, 205b, and the membrane layer 202a, 202b are to be subjected to the plasma treatment. Then the contact area, as described above, not subjected to the plasma treatment will also not be bonded together. During the plasma treatment, the contact area will be covered by a piece of Al foil. When fluid pressure is applied to each check valve 106a, 106b, the membrane layer 202a, 202b deforms and becomes detached from the top layer 204a, 204b. More specifically, the first and second check valves 106a, 106b ensure that fluid can enter the pump 104 through only check valve 106a, 106b at any time, and also exit the pump 104 through the other check valve 106a, 106b. That is, depending on fluid pressure conditions, fluid enters the pump 104 through the first check valve 106a, and exits the pump 104 through the second check valve 106b, or vice-versa when the fluid pressure conditions are reversed.

Due to the elasticity of PDMS, when the bottom of the pump 104 is pressed and deformed, fluid pressure in the pump 104 increases consequently since the fluid in the pump 104 is now contained within a smaller volume space. For the first check valve 106a, the high fluid pressure in the pump 104 is exerted into the associated fluid chamber 208 (through the hole in the associated membrane layer 202a) and so causes the said fluid chamber 208a to expand. Thus the membrane layer 202a is consequently pushed upwards to act tightly against the protruding portion 205a of the top layer 204a, sealing the fluid channel of the first check valve 106a, i.e. the first check valve 106a is switched off. For the second check valve 106b, the high fluid pressure in the pump 104 pushes and then deforms the associated membrane layer 202b of the second check valve 106b, which causes the protruding portion 205b of the top layer 204b (of the second check valve 106b) to be detached from the membrane layer 202b. This opens the fluid channel of the second check-valve 106b. The second check valve 106b is switched on, and fluid in the pump 104 passes through.

Continuing, when pressing on the bottom layer of the pump 104 is released, the pump 104 recovers to its initial shape, being resiliently deformable. Thus the fluid pressure in pump 104 now conversely becomes low, and in turn causes the membrane layer 202a of the first check valve 106a to be downwardly deformed. So the first check valve 106a is now switched on and external fluid may be drawn in and introduced into the pump 104. Meanwhile the low fluid pressure in pump 104 causes the associated membrane layer 202b of the second check valve 106b to have an upward deformation, which seals the fluid channel of the second check valve 106b. So the second check valve 106b is now switched off. In summary, one check valve 106a, 106b is switched on, when another check valve 106a, 106b is switched off, based on fluid pressure conditions imposed by pressing on, or releasing the pressing of the pump 104. So, a one directional fluid flow is imposed by pressing the elastically deformable pump 104, as above described.

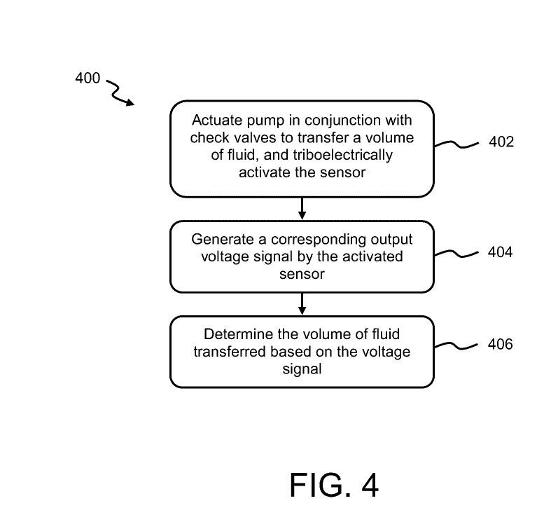

FIG. 4 is a flow diagram of a method 400 of using the device 100. Particularly, the method 400 comprises, at step 402, actuating the pump 104 in conjunction with the first and second check-valves 106a, 106b to transfer a volume of fluid (to the fluid outlets 108), and to cause at least a portion of the sensor 102 to be triboelectrically activated according to an amount of deformation of the pump 104; and, at step 404, generating a corresponding output voltage signal by the activated sensor 102 for enabling the volume of fluid transferred by the deformed pump 104 to be determined. Lastly, the volume of fluid transferred by the deformed pump 104 is determined at step 406, which is an optional part of the method 400.

### 2. Results and Discussion

### 2.1 Optimization and Characterization of Surface Materials of the Triboelectric Layer Pair

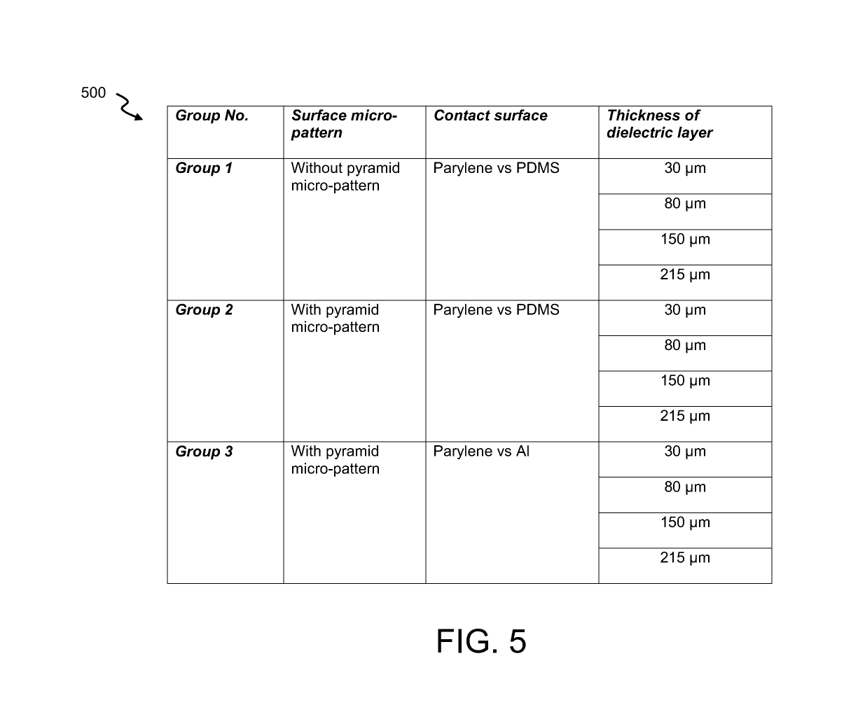

To enhance the sensitivity of the sensor 102 (for delivery volume detection), the output voltage generated by the TEH is expected to be as high as possible. So effects of the surface micro-features/patterns, material of contact surface and a thickness of the PDMS dielectric layer are investigated and evaluated. Three groups of test samples (i.e. Group 1, Group 2, and Group 3) with different PDMS thickness are prepared as shown in a tabulation table 500 depicted in FIG. 5.

In the evaluation tests, TEH patches were fixed onto a force gauge and applied onto the PDMS or Al contact surface with a same force, which is about 10 N. To make the open circuit output voltage of the tested TEH patches reaches the maximum value possible, the surface of the TEH patches is fully contacted with the PDMS or Al surface.

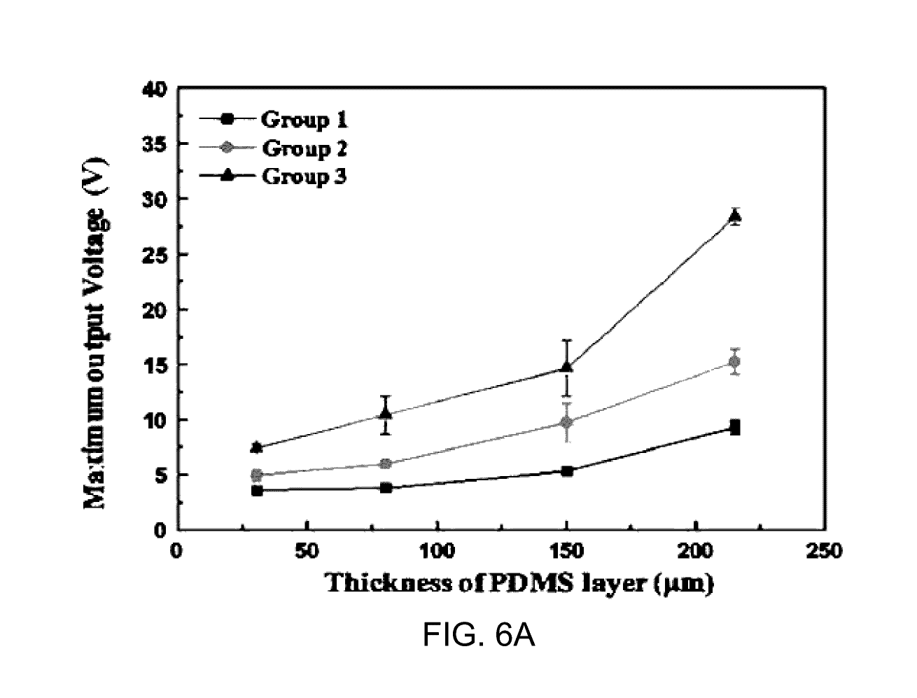

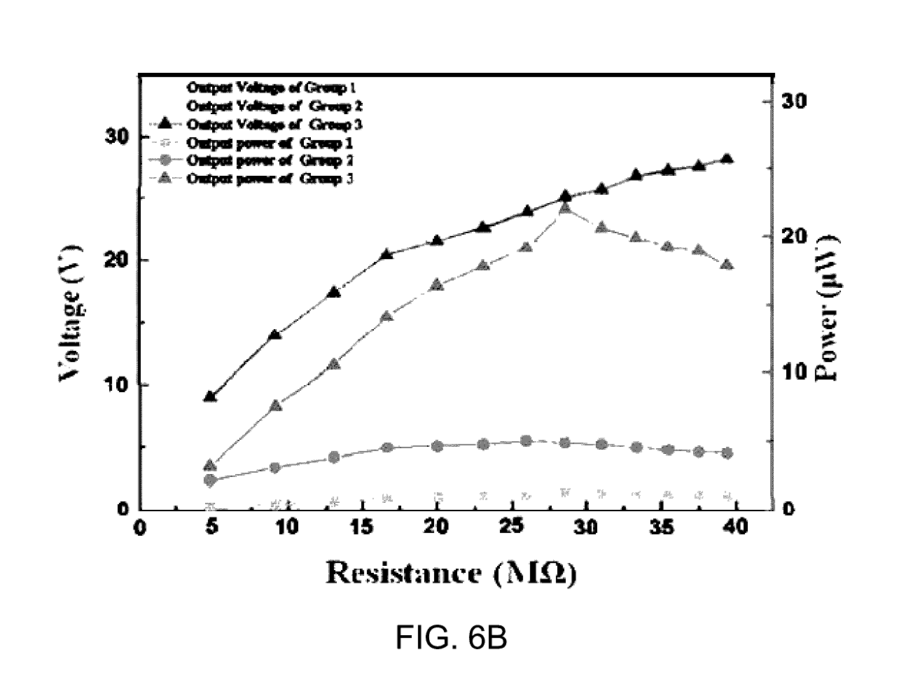

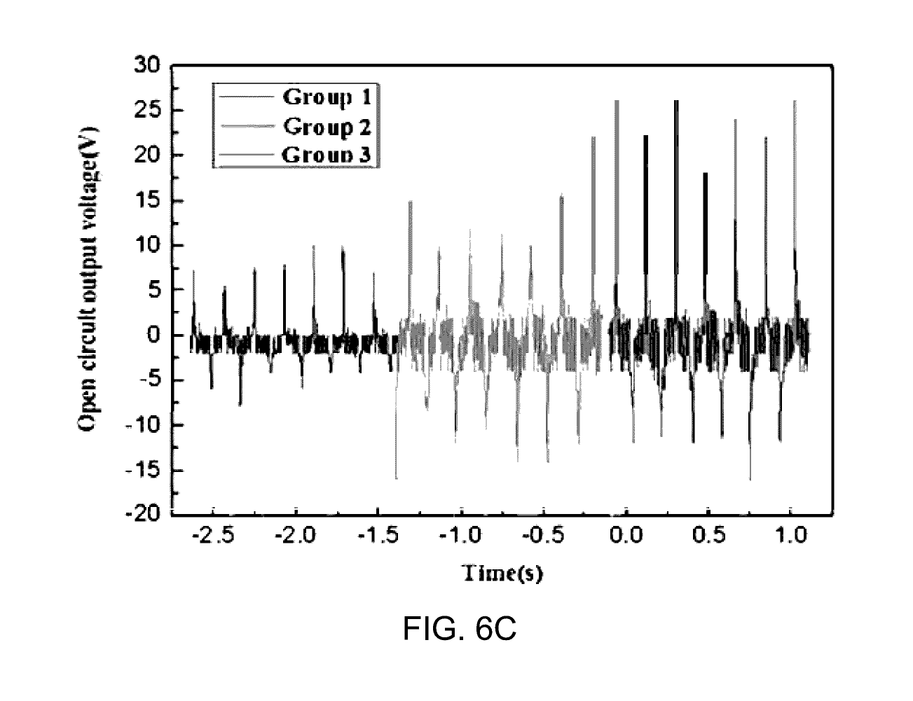

The experimental measurement data obtained is shown in FIG. 6A. According to the V-Q-x relationship for contact-mold TEHs, the output voltage, V, is determined by the following equation:

V=E_{dielectric}×d+Eₐᵢᵣ×x (1)

where E_{dielectric} is the electric field through the dielectric layer generated by the tribo-charges on the opposite sides of the TEH; d is the thickness of the dielectric layer; Eₐᵢᵣ is the electric field through the spacing between the top surface of TEH and contact surface, this electric field is generated by the tribo-charges on the TEH surface and contact surface; x is the spacing between the TEH surface and contact surface (i.e. the air gap 116). For ideal fully contact-mold TEHs, the output voltage should theoretically increase with the increase of thickness of the dielectric layer.

For Group 1 of the test sample, there are no micro-features/patterns formed on the surface of the TEH. The two contact surfaces are formed of parylene and PDMS. It can be seen that the output voltage increases from 3.8 V to 9.5 V when the thickness of PDMS dielectric layer increases from 30 μm to 215 μm—an observation which is consistent with equation (1).

For Group 2 of the test sample, there are micro-features/patterns (i.e. pyramid shaped) formed on the surface of the TEH. The material configurations of the two contact surfaces are same as in Group 1. Compared with Group 1, the output voltages of the test samples of Group 2 have about 50% enhancement with the same PDMS thickness. The output voltage increases from 5.2 V to 14.6 V when the thickness of the PDMS dielectric layer increases from 30 μm to 215 μm. Thus the micro-features/patterns on the surface of TEH can beneficially enhance the output voltage generated by 50%.

The test samples of Group 3 are the same as Group 2. To further increase the output voltage generated, the contact surface used is changed from PDMS to Al. Since Al is more triboelectrically positive than PDMS, these contact surfaces of parylene and Al are able to generate a higher output voltage. For the test samples of 30 μm, 80 μm and 150 μm, the output voltages are about 50% higher than their counterparts of Group 2. For the test sample having a thickness of 215 μm for the PDMS dielectric layer, the output voltage is enhanced by 100% compared to the test sample with the same thickness in Group 2. This maybe induced by the relatively thick layer of PDMS. During evaluation, not only do the micro-features/patterns get deformed, but the PDMS layer itself also suffered serious deformation which induced more charge transport. Thus enhancement of the output voltage generated by thicker PDMS dielectric layer is much higher than that of thinner PDMS dielectric layer.

The generated output power for each group obtained by changing the load resistance is shown in FIG. 6B. The change of the maximum output power for each group appears to follow the same trend as that of open circuit output voltage shown in FIG. 6A. FIG. 6C shows output voltage over time of the different triboelectric surface pairs when the thickness of the PDMS layer is 200 μm.

### 2.2 Optimization and Evaluation of the Triboelectric Sensor

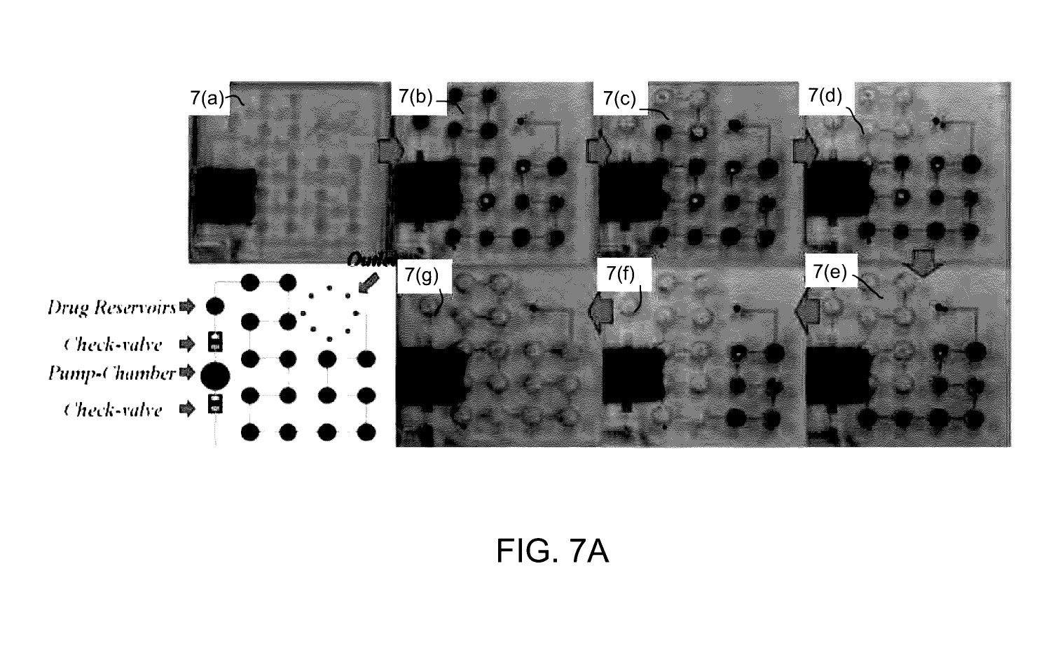

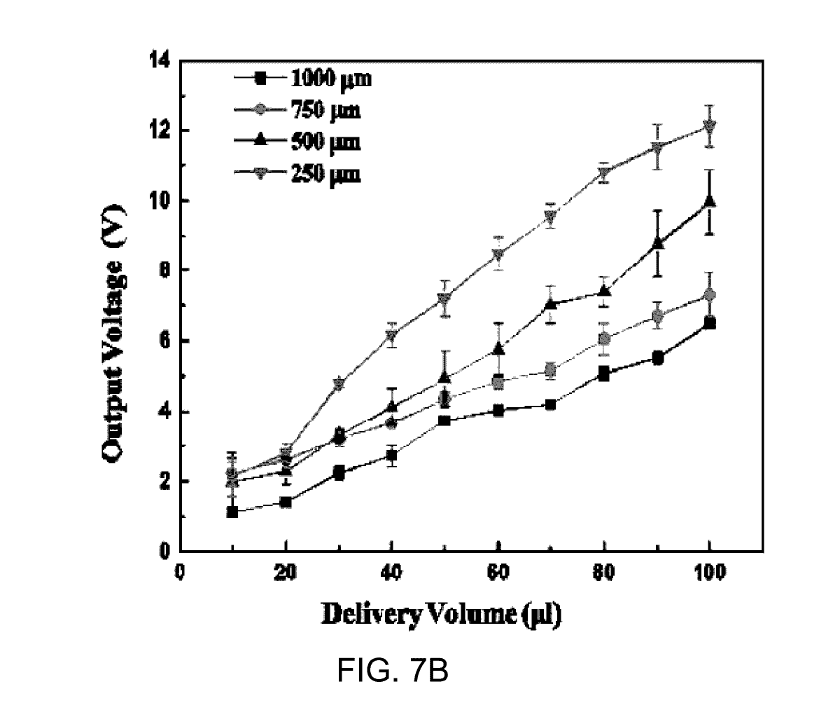

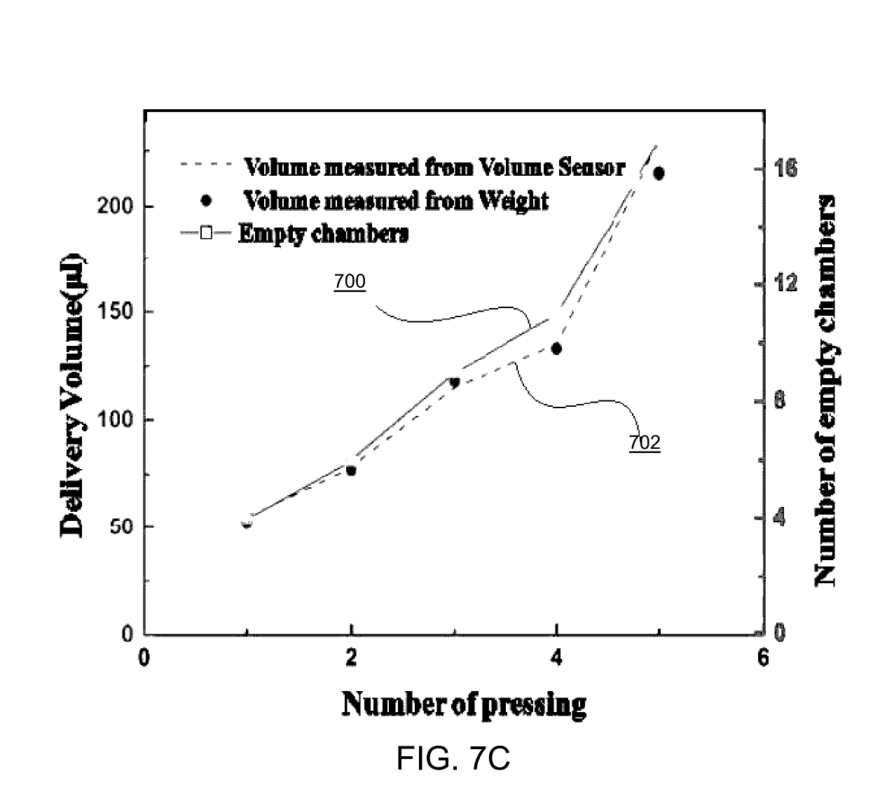

In equation (1), the spacing “x” (i.e. the air gap 116) between the top surface of the TEH and another contact surface is another parameter to determine the magnitude of output voltage generated. So to investigate the effect of the spacing between the TEH surface and contact surface, which is the height of the PDMS chamber, the spacing is changed from 250 μm to 1000 μm. To clarify, the PDMS chamber refers to the sensor 102, in which the first and second portions 112, 114 respectively form the top and bottom surfaces of the PDMS chamber. For this test, the TEH samples are formed with pyramid-shaped micro-features/patterns on the surface and having PDMS dielectric thickness of 215 μm, which are the test samples generating the highest output voltage previously depicted in FIG. 6A. The TEH samples to be tested were assembled above the pump 104, similar to the configuration shown in FIG. 1A. The pump 104 was pressed to deliver a certain volume of liquid within a range from 0.01 ml to 0.1 ml. The output voltage generated was recorded and the relationship between the delivery volume and output voltage is shown in FIG. 7B.

According to equation (1), for fully contact-mold TEHs, the output voltage generated increases with increase in the spacing “x”. But as shown in FIG. 7B, the output voltage generated instead decreases with increase in the spacing “x”. This is because the TEH shown in FIG. 1A is not working in fully contact-mold. With the same deformation of the top layer of the pump 104, the contact area between the first and second portions 112, 114 actually decreases with increase in the spacing “x”. Although the TEH is able to generate higher output voltage when the spacing “x” is narrower, the standard deviation was also higher, which is shown by the error-bar in FIG. 7B. Thus there is a trade-off between the accuracy and generated output voltage of the sensor 102. When the pump 104 is manually pressed by finger, the TEH patch deforms and contacts with the Al surface. However, these two surfaces not only contact with each other but also squeeze each other and further have friction generated by the contact between them. So the entire TEH not only works in contact mode but also partially works in friction mode. The friction will thus also contribute to the output voltage generated. The amount of friction generated is more pronounced when the spacing “x” is narrower. This is because with a same force applied on the pump 104, the TEH patch squeezes the Al surface more when the spacing “x” is narrower. If the force applied onto the pump 104 is not perpendicular to the Al surface, then friction is generated. However, the direction of the force applied by human finger cannot be too well controlled. Thus, the extent of resultant friction generated also cannot be well controlled. This is the reason why the error bar is larger when the spacing “x” is narrower. When the spacing “x” is defined to be 1000 μm, friction is almost not generated because deformation of the TEH surface is not large enough to squeeze the Al surface. Based on the data when the spacing “x” is defined as 1000 μm, the Coefficient of Variation is % CV=11%. To have a better accuracy for the delivery volume monitoring, the design of 1000 μm spacing was therefore applied to test samples in other evaluation tests.

A demonstration of drug delivery with volume sensor monitoring, using a patch which incorporates the first device 100, is shown in FIG. 7A. The weight of the patch was measured after each pressing to calculate the volume of the liquid delivered, as shown in 7(a) through 7(e) in FIG. 7A. Drug delivery means delivery of fluid drug medications. The output voltages generated by the sensor 102 were recorded and converted to delivered volume according to the voltage-volume curve calibrated in FIG. 7B. Moreover, a number of empty drug reservoirs 110 were counted to roughly estimate the delivered drug volume. The results of the delivery volume of each finger-pressing measured by these three methods are shown in FIG. 7C. The number of empty drug reservoirs 110 for each pressing made is 4, 2, 3, 2 and 6, which is indicated by a first line labelled with reference numeral 700 in FIG. 7C. The delivered volume of the drug as measured by weight and by the sensor 102 are respectively indicated as bold spots and by a second line labelled with reference numeral 702 in FIG. 7C. Specifically, the delivered volume measured by the sensor 102 and by weight is almost about the same except for the last data point, i.e. the volume measured by the sensor 102 is a bit higher than the volume measured by weight. The applied force recorded by the sensor 102 at the last finger-pressing data point was higher than the required force to deliver the rest solution in the patch, where the accurate volume is characterized by weight. It is to be appreciated that the rest solution is of 123 μL for the last pressing, but the force detected from the sensor 102 is able to deliver about 138 μL of solution. Secondly the delivered volume measured by counting the empty drug reservoirs 110 does not perfectly match the data measured by the sensor 102 and by weight, because there was still some residual solution in the empty drug reservoirs 110 and associated interconnecting fluid channels. Generally, the delivered volume of drug can straightforwardly be estimated by counting the number of empty drug reservoirs 110. Thus the sensor 102 is useful for determining relatively precise delivered volume, since certain usage scenarios may require information pertaining to accurate delivered volume.

### 2.3 Development of the Lab-on-Chip Drug Delivery System

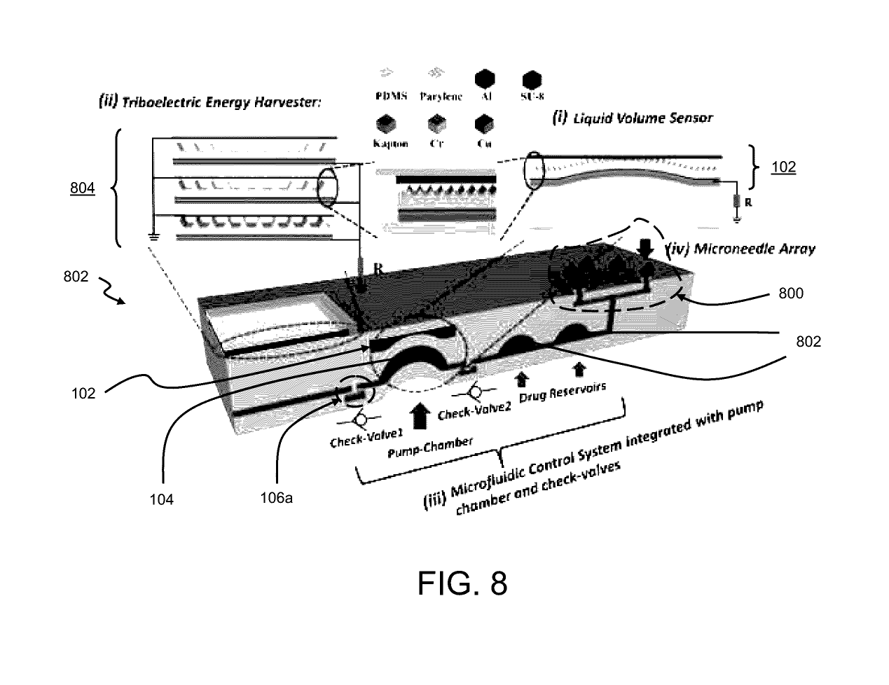

Further, the first device 100 is integrated with a micro-needle array device 800 to form a lab-on-chip (LoC) drug delivery skin patch 802, shown in FIG. 8. Detailed description for the micro-needle array device 800 will be described in the second embodiment below. To make the skin patch 802 able to maintain a tight and stable contact with skin surfaces (which are contoured and deformable), the entire skin patch 802 is made of PDMS, a well-known biocompatible soft and flexible material. So, the skin patch 802 is arranged to be flexible and can easily adhere onto skin surfaces. Particularly, the micro-needle array device 800 is incorporated at where the fluid outlets 108 originally are on the first device 100 for enabling skin penetration, and thus the skin patch 802 is usable for transdermal drug delivery.

On the top surface of the skin patch 802, a plurality of patches of TEH 804, each being of 2×2 cm² area, in a stacked arrangement is further integrated, which leverages the similar structure of the sensor 102 to enable further integration of active power components with the skin patch 802. Examples of the power components include an integrated circuit, a microprocessor, and a LCD reading panel. In addition, the skin patch 802 may further comprise a convertor circuit (not shown) configured to convert collective output voltages generated by the patches of TEH 804 into electricity for powering the power components. It is to be appreciated that the configured area of each patch of TEH 804 may also be of other sizes, depending on requirements of different applications intended for the skin patch 802. By pressing the TEH 804 from the top of the skin patch 802, electrical power can be generated (as explained). The skin patch 802 also incorporates first, second, and third dry adhesive patches (not shown) to make the skin patch 802 be easily fixed onto a curved skin surface on a user (e.g. a patient), i.e. the micro-needle array device 800 is arranged between the first and second dry adhesive patches and the TEH 804 is then arranged between the second and third dry adhesive patches. It is to be appreciated that two methods may be used to generate electrical power from the TEH 804 by applying the skin patch 802 onto different suitable body locations of a user. When the skin patch 802 is adhered to the elbow of a straightened forearm, the spacing between any two dry adhesive patches is slightly shorter than the patch length of the TEH 804. Thus the TEH 804 is bent and not in contact with the skin surface in this initial position. When the elbow is subsequently bent, the TEH 804 is stretched and comes into contact with the skin surface at the elbow. Thereafter, when the bent elbow is straightened, the spacing between the second and third dry adhesive patches is compressed to cause the TEH 804 to be bent and be separated again from the skin surface. Electrical power may thus be generated and harvested from the skin patch 802 by repeating the above described sequence of simple steps.

For the case in which the skin patch 802 is applied onto a flat skin surface like the arm or abdomen, electrical power can be generated by pressing and releasing the TEH 804 to induce contact and separation between the TEH 804 and skin surface. However, due to the sticky surface of PDMS, once the triboelectric contact surface is pressed onto the skin surface, the skin patch 802 cannot automatically be separated from the skin surface when the pressing is released. So to solve this problem, a fourth dry adhesive patch (not shown) is assembled at the reverse side of the TEH 804. Accordingly, when the finger lifts up, the fourth dry adhesive patch is able to provide a pulling force to cause the TEH 804 be detached from the skin surface. Because the adhesive force provided by the fourth dry adhesive is limited, the fourth dry adhesive detaches from the finger when lifted up to a certain height. In order to have a maximized output power generated by the TEH 804, the fourth dry adhesive is optimized to provide a maximum lift-up height.

### 2.4 Fabrication of the Skin Patch

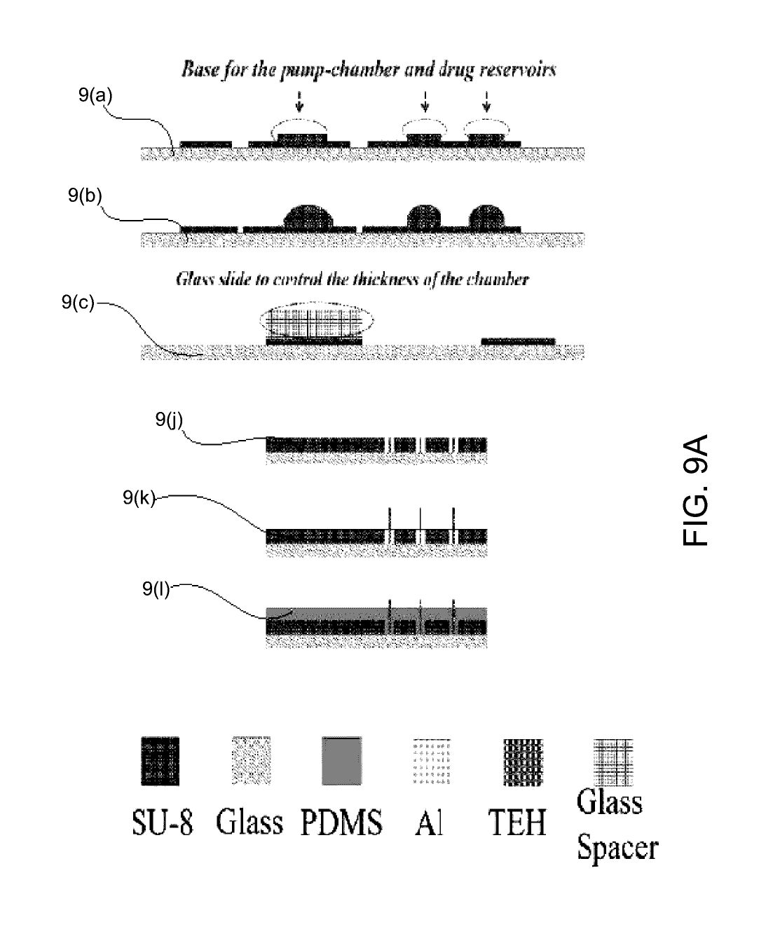

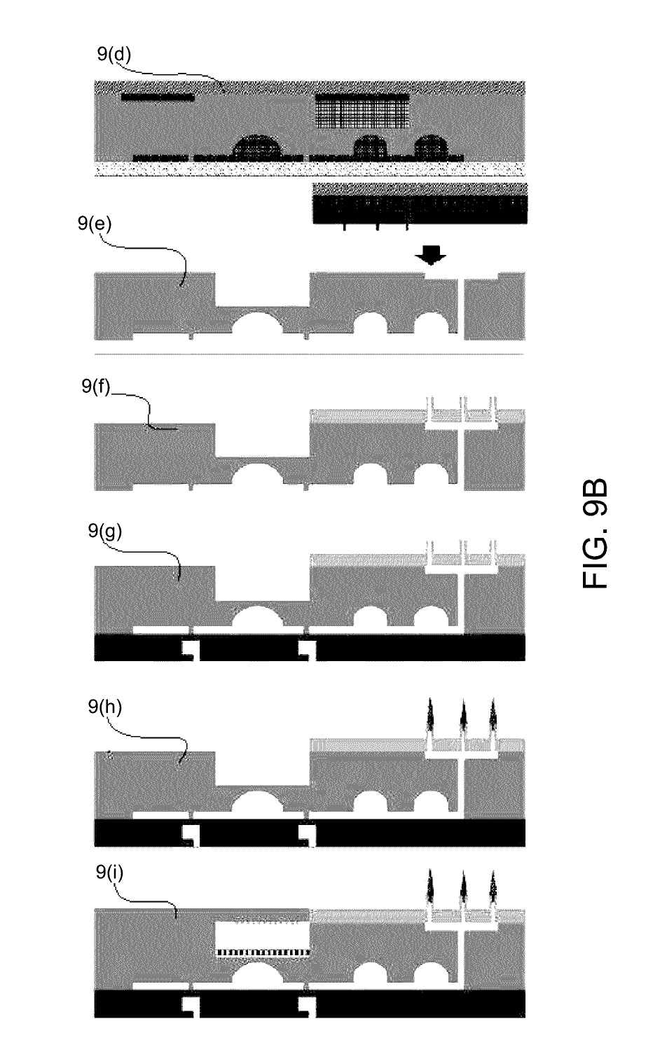

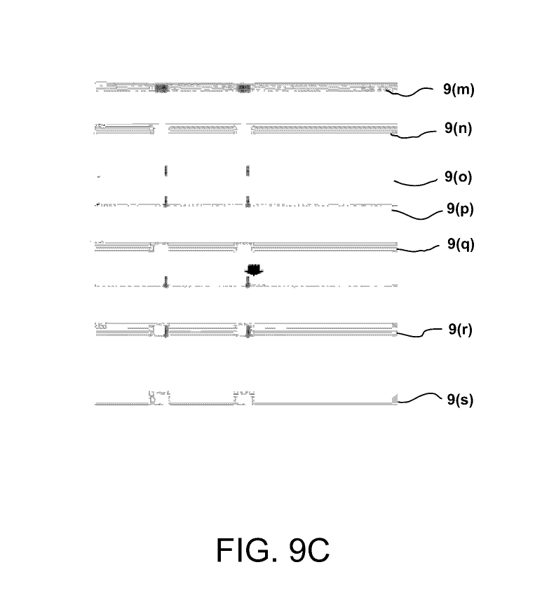

The detailed fabrication process of the skin patch 802 is shown in FIGS. 9A-9C. To begin, first and second SU-8 photoresist molds (not shown) are to be prepared. The first SU-8 mold is for fabricating the upper layer of the check valves 106a, 106b, pump-chamber 105, drug reservoirs 110 and fluidic channels shown as 9(a) and 9(b) in FIG. 9A. A SU-8 layer is coated and patterned on a glass wafer to form a SU-8 base, as illustrated in 9(a) in FIG. 9A. On the first SU-8 mold, there are to be bases for forming the pump-chamber 105 and drug reservoirs 110. Then SU-8 in liquid phase is introduced onto the SU-8 base of 9(a) in FIG. 9A to form SU-8 hemispheres (i.e. 9(b) shown in FIG. 9A). Next, the sample in 9(b) shown in FIG. 9A is baked to solidify the SU-8 hemispheres, with the baked sample further exposed to UV light and then post-baked to make the SU-8 base and hemispheres crosslink with one another, thereby forming the first SU-8 mold.

Meanwhile, the second SU-8 mold is for fabricating fluidic channels for the micro-needle array device 800 and the PDMS chamber of the sensor 102—see 9(c) shown in FIG. 9A. To have good control in fabricating the spacing of the chamber of the sensor 102, a glass slide of a certain thickness is attached onto the base of the chamber, thereby forming the second SU-8 mold. Then the first and second SU-8 molds shown as 9(b) and 9(c) in FIG. 9A respectively are face-to-face aligned and then fixed with a spacer at the edge. The intervening space between the first and second SU-8 molds is filled with PDMS, shown as 9(d) in FIG. 9B. The first and second SU-8 molds now fixed together, with the layer of PDMS filled in between, are kept at room temperature for 24 hours without baking to avoid air bubbles formation within the PDMS layer. Then the two fixed SU-8 molds are cured at 75° C. for 30 minutes. The cured sample is cooled to room temperature and the two SU-8 molds are then released from the PDMS layer, shown as 9(e) in FIG. 9B. The resulting PDMS layer has patterns on both opposing faces and is to be used as the main body of the skin patch 802. The top face of the main body has formed channels for interfacing with the micro-needle array device 800 whereas the bottom face of the main body has formed patterns for implementing the microfluidic control system (i.e. the pump 104 and the first and second check valves 106a, 106b).

To fabricate the micro-needle array device 800 on the top face of the PDMS layer, a third SU-8 mold is to be prepared (i.e. 9(j) through 9(l) shown in FIG. 9A). For the third SU-8 mold, a first SU-8 layer with an array of holes is patterned is shown as 9(j) in FIG. 9A. The holes are for forming four-beam PDMS pillars (to be elaborated in the second embodiment in Section 4). Then a pillar array is aligned to the patterned array of holes as a second SU-8 layer, thereby forming the third SU-8 mold. The second SU-8 layer is for providing a vertical channel connecting the channel array on the top face of the main body of 9(e) in FIG. 9B with the micro-needle array device 800, which will be assembled later. Then a layer of PDMS is coated onto the third SU-8 mold (shown as 9(k) in FIG. 9A) to fill the hole pattern and form a whole layer above the third SU-8 mold—see 9(l) in FIG. 9A. The thickness of the PDMS layer (i.e. 150 μm) is smaller than the thickness of the height of the SU-8 pillar (i.e. 350 μm). Thus the top section of the SU-8 pillar is not covered by the PDMS layer. A through hole is created in the third SU-8 mold through the PDMS layer to form a vertical channel inside the micro-needle array device 800. The sample in 9(l) is then arranged bottom-side up, and bonded to the top face of the PDMS layer of 9(e). With this, the third SU-8 mold is released—see 9(f).

Now at the top face of the main body, a PDMS pillar array is aligned and connected to the channel array. Then for the bottom face of the main body, two PDMS layers are needed to form the two check valves 106a, 106b. Thus fourth and fifth SU-8 molds are additionally required. 9(m) shows the fourth SU-8 mold for forming the bottom chambers of the check valves 106a, 106b. A thick layer of PDMS layer is coated onto the fourth SU-8 mold, subsequently cured, and the cured PDMS is released from the fourth SU-8 mold, as shown in 9(n). Next, 9(o) shows the fifth SU-8 mold for forming the holes on the membrane layer 202a, 202b of the check valves 106a, 106b. Then a thin layer of PDMS is coated onto the fifth SU-8 mold—see 9(p). To create through holes on the membrane layer 202a, 202b, the thickness of the PDMS (i.e. 40 μm) is to be smaller than the height of the SU-8 pillar (i.e. 350 μm). Thereafter, the PDMS layer in 9(n) is aligned with the now PDMS-coated fifth SU-8 mold of 9(p) (i.e. refer to 9(q)) and bonded together as depicted in 9(r). Then the bonded PDMS layers are released from the fifth SU-8 mold as depicted in 9(s). The bonded PDMS layers have the membrane layer 202a, 202b with holes and chambers for implementing the check valves 106a, 106b. The PDMS layers are aligned and bonded to the bottom face of the main body as per 9(g). Then sharp tips (for the micro-needle array device 800) are assembled onto the PDMS pillar array as shown in 9(h). The sharp tips assembly is realized by double drawing lithography—see Section 4. Then the TEH is fixed at the bottom of the pump 104 to form the sensor 102, and a PDMS with a layer of Al coating is bonded above the TEH to seal the chamber of the sensor 102, as shown in 9(i).

### 2.5 Characterization and Optimization for TEHs in a Stacked Arrangement

Due to the small area of the skin patch 802, the power generated by a single layer of TEH will be limited. So, as described previously, the plurality of patches of TEH 804 in a stacked arrangement is adopted to enhance the output voltage generated. Particularly, multiple TEHs (of N layers, N>1) are stacked layer by layer, and connected in parallel to achieve an N-times charge transfer effected by each round of pressing, as opposed to using a single layer of TEH. If all the N-layered TEH has the charge transport simultaneously, the total transferred charge will increase N times. Meanwhile, due to the parallel connection of all the layers in the N-layered TEH, the total inner resistance will decrease, which further enhances the output power.

Further due to reliability concerns (referring to FIGS. 9A-9C), a parylene coating on the bottom PDMS dielectric layer is used to protect the metal layer at the backside in the proposed design as shown in FIGS. 9A-9C. An experiment was conducted for the TEHs without the parylene coating as a comparison against previous data with parylene coating for characterizing the impact of having the parylene coating. Briefly, results pertaining to load resistances of peak powers are the same for both TEH with and without the parylene coating. However, the peak powers attained by TEH without the parylene coating are enhanced by about 27% on average. This suggests that, if the backside metal layer of the TEH patch is not easily detached or scratched, the parylene coating need not be applied, since the PDMS surface may instead give better performance.

### 3. Summary

Triboelectric energy harvester has been applied to various kinds of wearable sensors and electronics for its flexible and thin film structure characteristic. The sensor 102 (incorporated in the first device 100) leveraging the triboelectric mechanism is proposed, and integrated within the (wearable) skin patch 802 to realize a manually-controlled large volume drug delivery function. Drug delivery is triggered by finger-pressing on the pump 104 of the first device 100. To power active components that may be integrated on the disclosed skin patch 802 in future, a stacked layer triboelectric energy harvester (TEH) design was studied and characterized. Increasing the number of stacked layers significantly enhances the output power generated, as found. The collective output power generated by a TEH with 3 stacked layers, with each layer of TEH configured to have an area of 2×2 cm², is 33 μW. Such electrical energy may be harvested even during drug delivery via finger pressing. It is to be appreciated that the harvested electrical energy may also be stored in a battery to provide required operation power for other active components or glucose sensors that may be integrated in the skin patch 802 for other desired applications.

The optimum pressing frequency (for actuating the pump 104) is about 2 Hz, which is within the reasonable range of usage scenarios based on manual finger pressing. The sensor 102 integrated within the skin patch 802 leverages largely a similar structure as the TEH 804. The air gap 116 between the triboelectric layer pair is optimized to be about 1000 μm for best sensing accuracy by the sensor 102. Thus, the delivery volume can be monitored, which is crucial in certain medical applications such as for insulin delivery, where the delivery dosage needs to be precisely controlled. Then, the micro-needle array device 800 is assembled onto the skin patch 802 to confirm drug delivery and volume monitoring functions by in vivo experiments in rats—see Section 6. The sensor 102 may also be integrated with other suitable drug delivery devices or lab-on-chip microfluidic devices where the liquid volume delivered needs to be accurately measured.

The remaining configurations/embodiments will be described hereinafter. For sake of brevity, description of like elements, functionalities and operations that are common between the different configurations/embodiments are not repeated; reference will instead be made to similar parts of the relevant configuration(s)/embodiments.

### 4. Micro-Needle Array Device

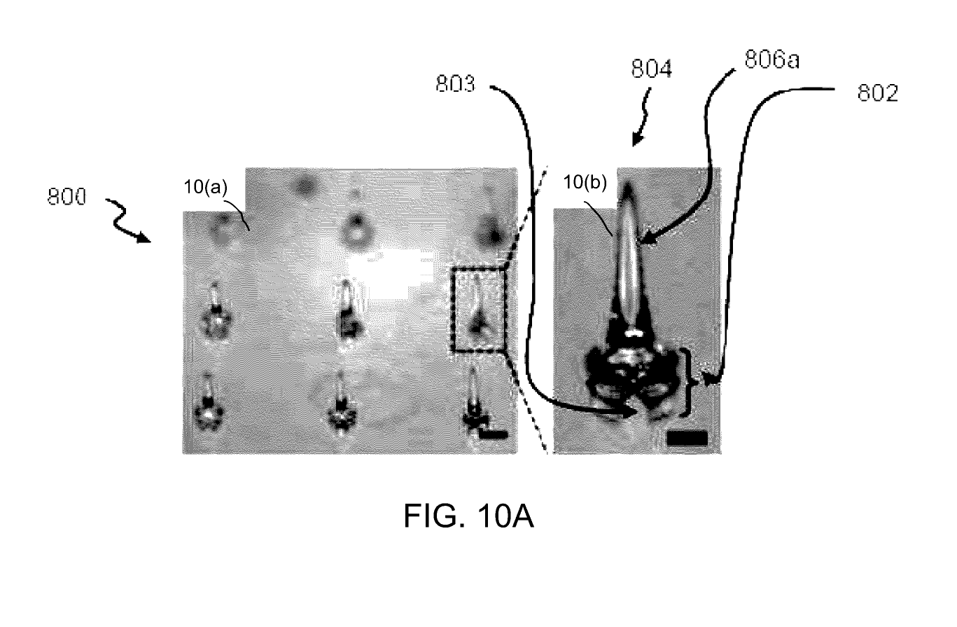

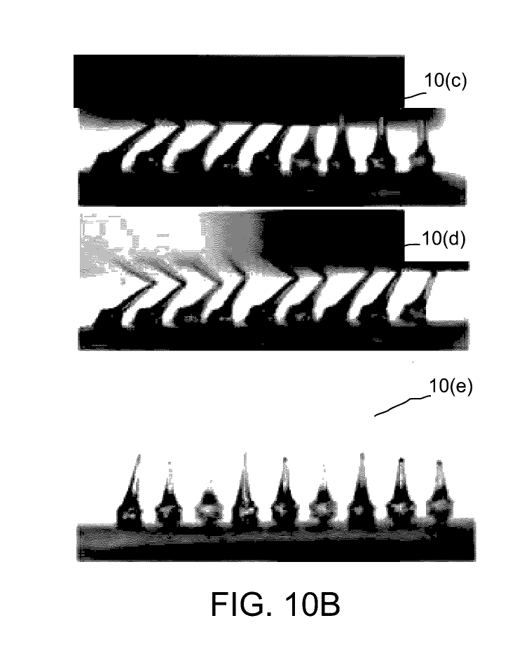

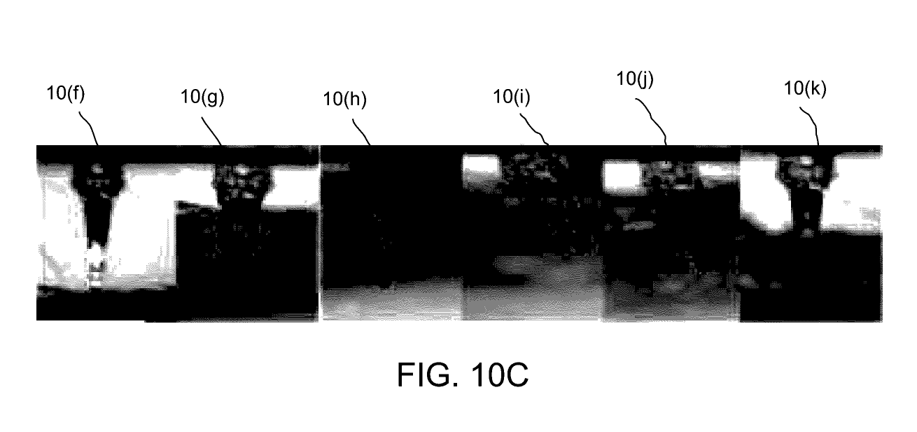

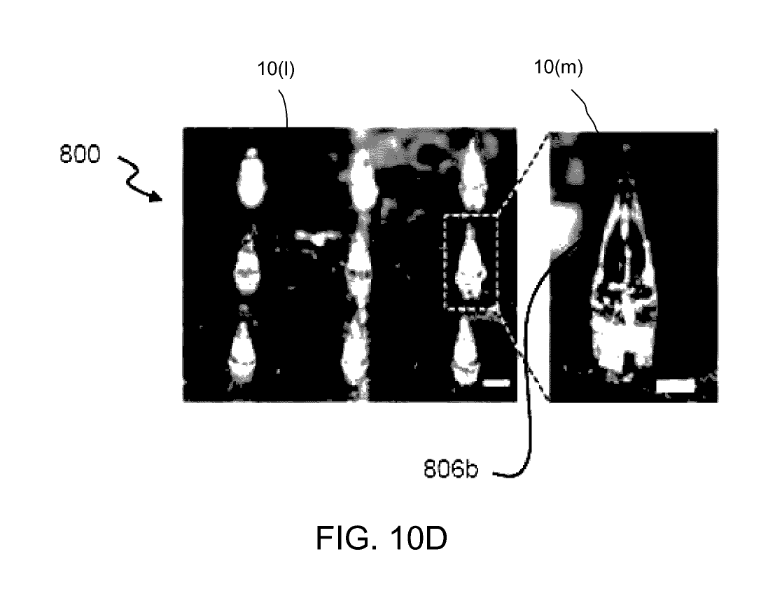

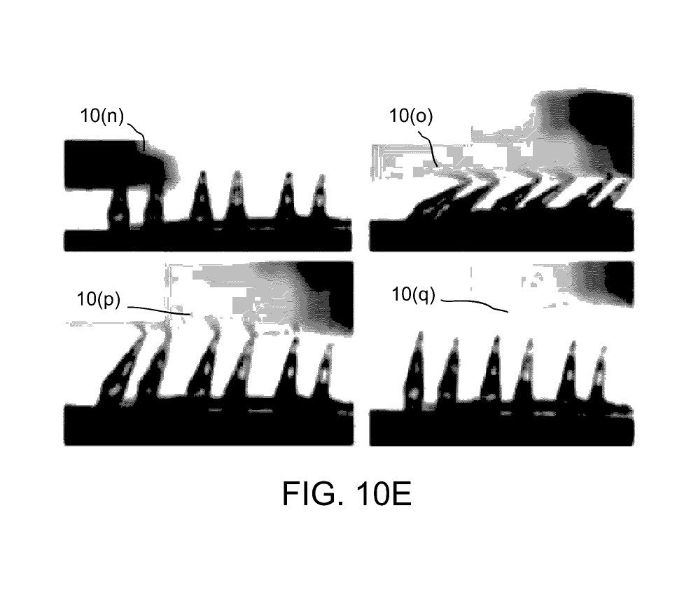

10(a) in FIG. 10A shows an optical image of the micro-needle array device 800 (which was aforementioned in Section 2.3), according to a second embodiment. The micro-needle array device 800 is hereafter abbreviated as the second device 800. Broadly, the second device 800 comprises a flexible substrate formed with a plurality of base protrusions 802 which are elastically deformable, each protrusion 802 having a plurality of recesses (or gaps) 803 for enabling fluid transfer; and a plurality of micro-needles 804 co-axially arranged on the respective protrusions, the micro-needles 804 being substantially rigid. When a lateral force is applied to the device 800, the micro-needles 804 are displaced off-axis relative to the protrusions 802 due to elastic deformation of the protrusions 802 caused by the force, and when the force is removed, the micro-needles 804 return to the co-axial arrangement.

More specifically, 10(b) in FIG. 10A also shows a magnified view of the structure of an individual bendable micro-needle 804 of the second device 800, in which the micro-needle 804 has a rigid sharp tip 806a arranged on protrusion 802 of the flexible substrate. Each protrusion 802 of the flexible substrate is formed as a bendable four-beam-pillar base in this instance, and thus referred to interchangeably hereafter. The sharp tip 806a is made of SU-8 photoresist in. 10(a) and 10(b) (thereafter “SU-8 sharp tip 806a”). For clarity, it is to be appreciated that 10(l) and 10(m) depicted in FIG. 10D are respectively similar to FIGS. 10A and 10B, except that the sharp tip of each micro-needle 804 is now instead made of maltose (thereafter “maltose sharp tip 806b”). There is also a recess 803 configured in between any two neighbouring beam-pillars of the four-beam-pillar base. The bendable four-beam-pillar base is made of PDMS with optimized stiffness to ensure a high success rate of skin penetration, while allowing a certain volume deformation. The SU-8 sharp tips 806a for skin penetration are assembled onto the four-beam-pillar base structure using double drawing lithography process. The gaps between the beam-pillars are partially filled with the same materials to form the micro-needles 804 during the drawing lithography step. Each four-beam-pillar base provides anchoring between the SU-8 sharp tip 806a and the flexible substrate in order to fix the SU-8 sharp tip 806a onto the flexible substrate and protect the SU-8 sharp tip 806a from breakage when an entire micro-needle 802 is bent during skin penetration.

To solve the needle breakage issue after skin penetration, a unique design for the bendable micro-needle 804 is proposed herein. As mentioned, the bendable micro-needle 804 is formed from the four-beam-pillar base made of PDMS and the relatively stiff SU-8 sharp tip 806a. The SU-8 sharp tips 806a are assembled onto the respective four-beam-pillar bases on the flexible substrate, each of which has four vertical gaps along the sidewalls of the four-beam-pillar (see 10(b)). The purpose of the four vertical gaps are twofold: (i). As the SU-8 sharp tips 806a are not bio-dissolvable, fluid drugs medication cannot be delivered to the skin without the gaps to enable a fluid transfer function from the micro-needles 804; and (ii). During the drawing lithography process, bottom portions of the respective SU-8 sharp tips 806a form a secure anchor in the associated gaps of the respective four-beam-pillar bases to enhance adhesion between the SU-8 sharp tips 806a and the respective four-beam-pillar bases. Due to the flexibility of the PDMS pillars, each micro-needle 804 bends when the lateral force is applied onto the micro-needle 804 exceeds the threshold (which is defined as the force required to make the micro-needle 804 bend, i.e. the buckling force). It is to be appreciated that bending of the micro-needles 804 means that the micro-needles 804 are displaced off-axis relative to the respective protrusions 802. The anchors of the SU-8 sharp tips 806a arranged in the gaps of the respective protrusions 802 secure the SU-8 sharp tips 806a onto the said protrusions 802, and prevent the micro-needles 804 being detached from the protrusions 802, when an entire micro-needle 804 undergoes bending during skin penetration. It is also to be appreciated that the foregoing described applies, mutatis mutandis, to the micro-needles 804 that are instead configured with the maltose sharp tips 806b, and hence not repeated for brevity sake.

FIGS. 10B and 10E show demonstrations of the bendable micro-needles 804 when a glass slide is used to push the micro-needle array device 800 from a lateral direction. FIG. 10B pertain to the micro-needles 804 arranged with the SU-8 sharp tips 806a, whereas FIG. 10E pertain to the micro-needles 804 arranged with the maltose sharp tips 806b. As can clearly be seen, the micro-needles 804 bend when the lateral force is applied. The micro-needles 804 recover to the original position (i.e. the co-axial arrangement) when the lateral force is removed. After skin penetration, relative movement between the skin patch 802 and the skin surface is inevitable as will be appreciated, which is a major reason for needle breakage. Advantageously, the deformable protrusions 802 at arranged on the bottom of and coupled to the proposed micro-needles 804 elastically deform to absorb the mechanical strain caused by lateral movement and further prevent breakage when the relative movement occurs. FIG. 10C are a series of photographs depicting occurrence of the relative movement between the skin surface and a micro-needle 804 after skin penetration, shown as 10(f) through 10(k). The dashed line shown in 10g-10j indicates the contoured profile of a position of the micro-needle 804 inside the skin after penetration. In 10(f)-10(h), the micro-needle 804 has penetrated the skin surface, and the entire sharp tip 806a, 806b is in the skin. In 10(i)-10(k), a lateral movement has occurred, and the micro-needle 804 is consequently displaced off-axis from the original position (i.e. the co-axial arrangement). The protrusion 802 (i.e. the four-beam-pillar base) arranged beneath the associated sharp tip 806a, 806b is elastically deformed to enable the sharp tip 806a, 806b to still remain within the skin (10(k)); when the micro-needle 804 is removed from the skin, the micro-needle 804 then recovers to the original position, without suffering needle breakage. The demonstration confirms that when the relative movement occurs, the micro-needles 804 bend to match the new position of the skin. On the other hand, if the relative movement is too large, then the micro-needles 804 are dragged out the skin and revert to the original position.

### 4.1 Characterization and Optimization of the Micro-Needles

Due to the elasticity of PDMS, the protrusions 802 are configured to bend when a force is applied on the micro-needles 804 exceed the buckling force. To realize a successful skin penetration, the stiffness of the protrusions is expected to be as high as possible. Thus, an evaluation study of the stiffness of the PDMS by varying a mix ratio of elastomer and curing agent is performed. Generally, the PDMS with a higher concentration of curing agent has a higher stiffness. Test samples with the mix ratio of 1:4, 1:6, 1:8 and 1:10 are evaluated for the micro-needles 804 having the SU-8 and maltose sharp tips 806a, 806b. Micro-needles 804 with the PDMS-based protrusions 802 and SU-8 sharp tips 806a were subjected to loading to study their mechanical stability. The variation of measured bending force versus displacement was recorded.

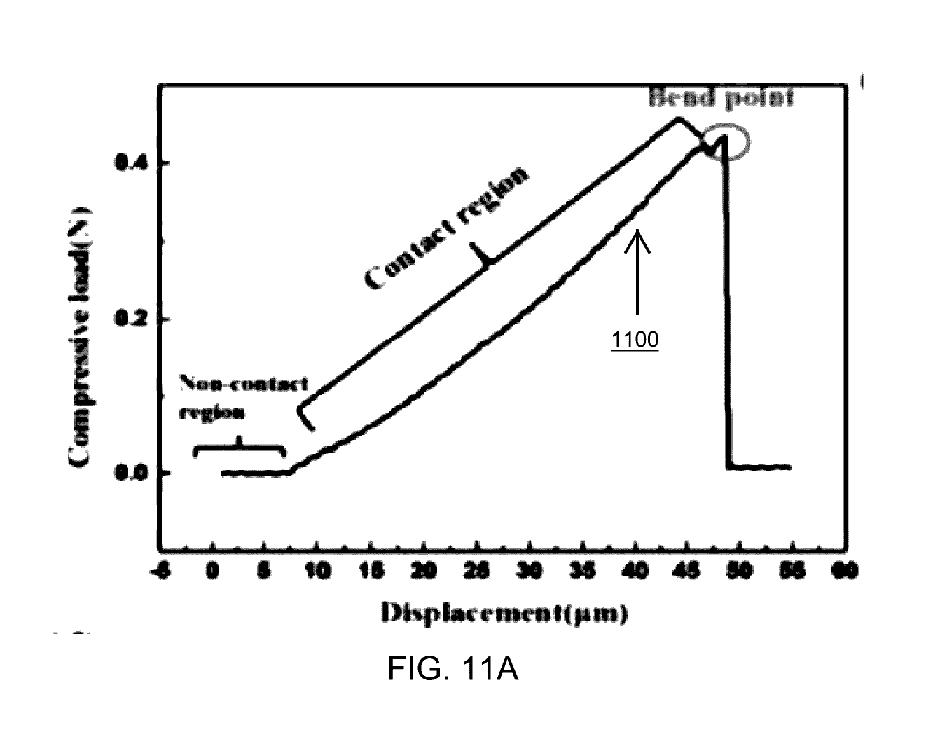

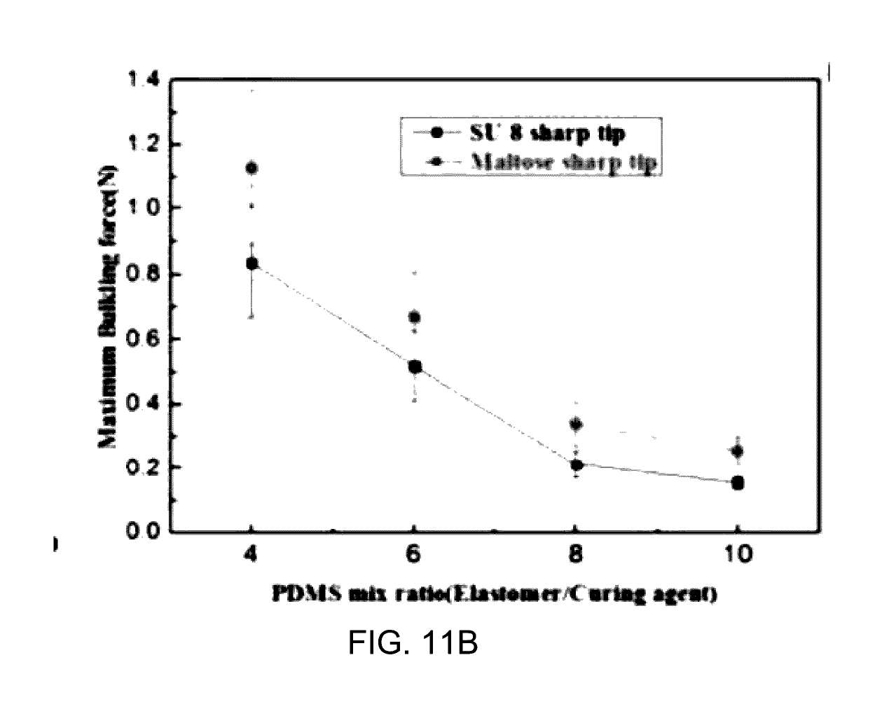

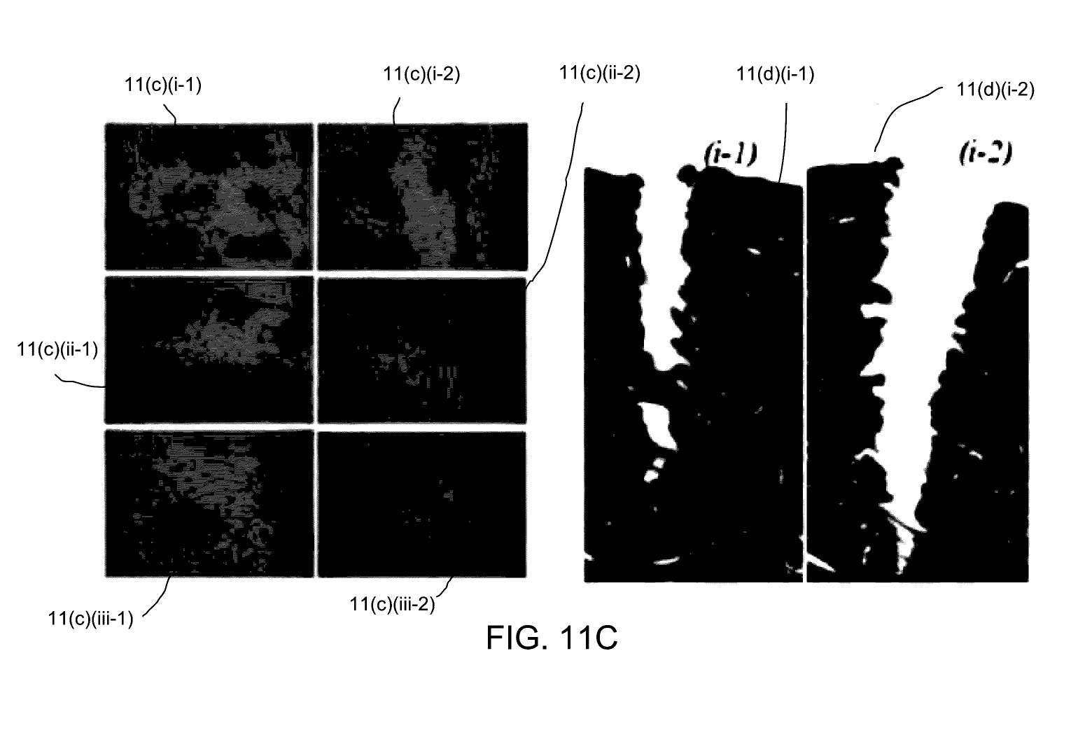

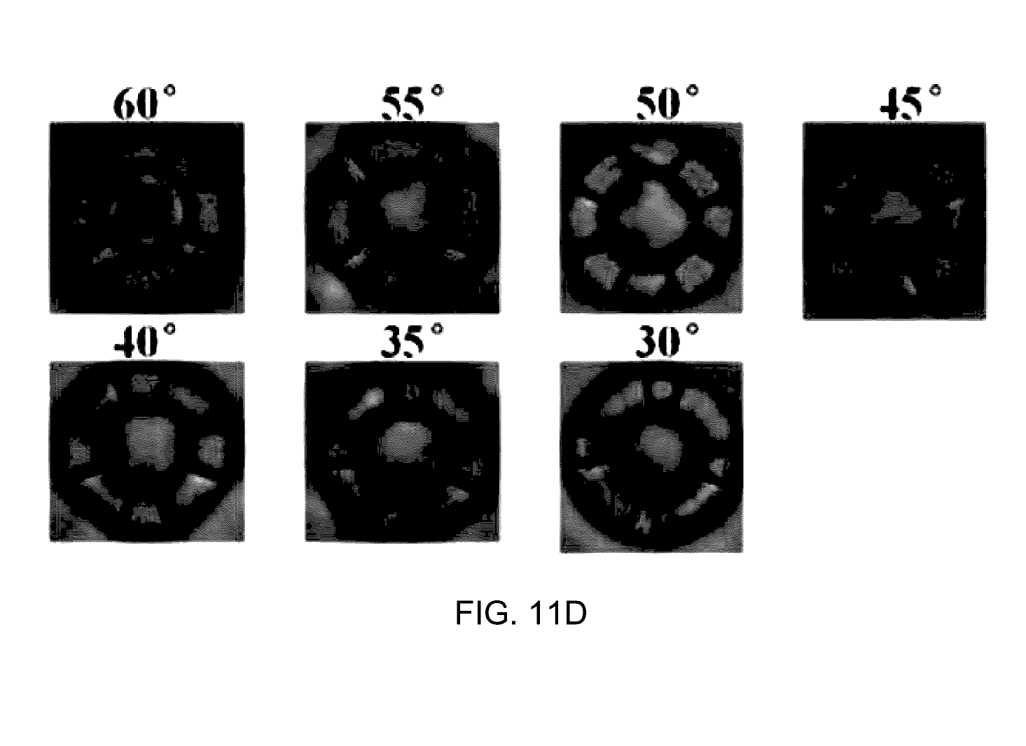

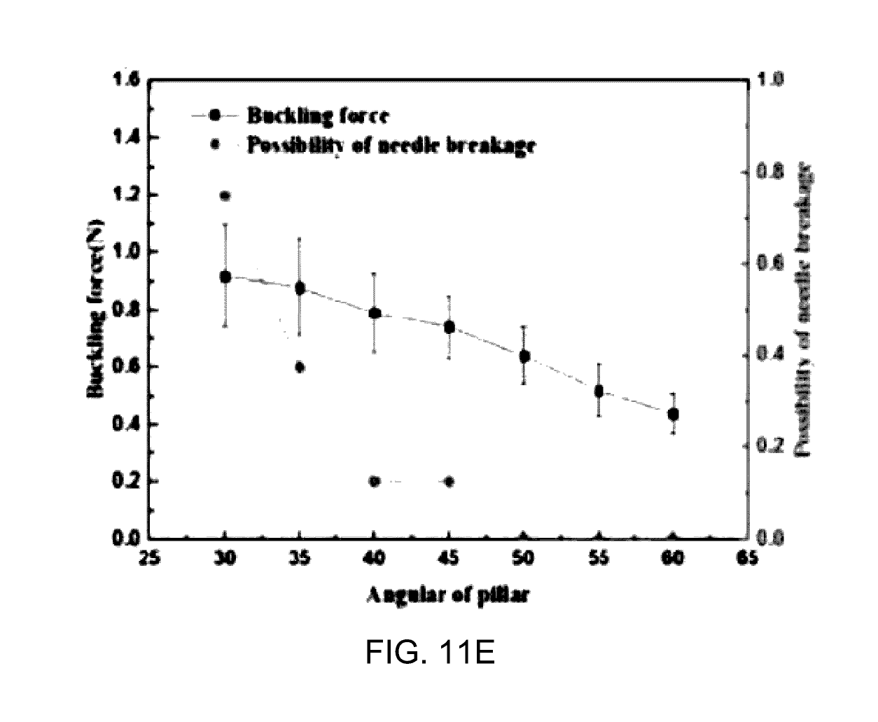

FIG. 11A shows a curve 1100 relating to measurement of the buckling force test for one test sample. There are three parts to the curve: a non-contact region, a contact region and a bend point, as indicated in FIG. 11A. The sharp drop of the force after the bend point confirms that the micro-needles 804 do not bend when the applied force is below the threshold. The forces of bend points of all the test samples with different mix ratio of PDMS are shown in FIG. 11B. For each mix ratio, ten micro-needles 804 were tested. As shown in FIG. 11B, for the mix ratio of 1:4, the bending force is about 0.8 N for SU-8 sharp tips 806a and 1.14 N for maltose sharp tips 806b. The bend force decreases with the decrease of the mix ratio. The buckling force of a micro-needle 804 with the maltose sharp tip 806b is higher than that of a micro-needle 804 with the SU-8 sharp tip 806a, because of the different shapes of the associated sharp tips. As shown in FIG. 10A, the micro-needle 804 with the SU-8 sharp tip 806a is slimmer than the micro-needle 804 with the maltose sharp tip 806b. This is because the viscosity of SU-8 is lower than that of maltose. During the double drawing lithography process, the SU-8 sharp tip 806a tends to have a slim central part while maltose sharp tip 806b tends to have a thicker and stronger central part. Thus the force to make the micro-needle 804 with the SU-8 sharp tip 806a bent is lower than that required for the micro-needle 804 with the maltose sharp tip 806b.

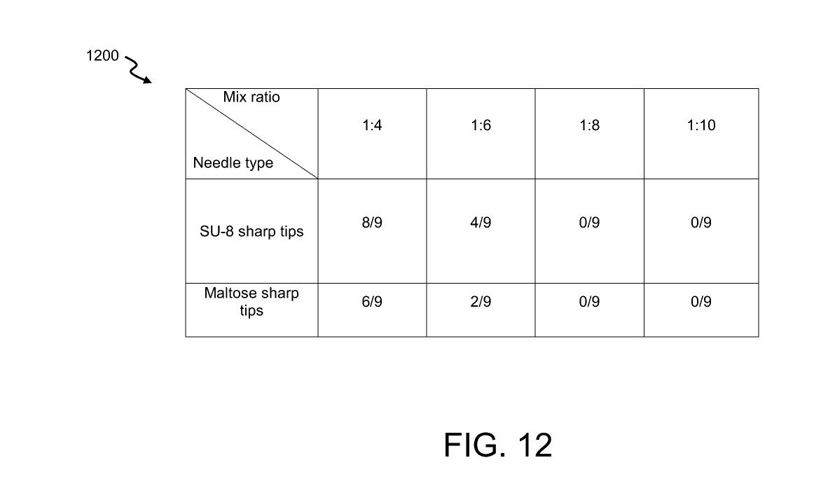

Skin penetration tests with samples of different PDMS ratio were also conducted. A 3×3 micro-needle array was applied onto a skin surface, and the number of penetrated holes created by the micro-needles 804 of the said 3×3 array was recorded. Accordingly, the results obtained are shown in FIG. 11C and recorded in the tabulation table 1200 of FIG. 12. For the test samples with a mix ratio of 1:4, there are eight (see 11(c)(i-1)) and six (see 11(c)(i-2)) penetrated holes made on the skin for micro-needles 804 with the SU-8 sharp tips 806a and maltose sharp tips 806b respectively. For the test samples with a mix ratio of 1:6, there are four (see 11(c)(ii-1)) and three (see 11(c)(ii-2)) penetrated holes made on skin for needles with the SU-8 sharp tips 806a and maltose sharp tips 806b respectively. For the test samples with a mix ratio of 1:8, there are no penetrated holes found on the skin for both micro-needle configurations, as shown in 11(c)(iii-1) and 11(c)(iii-2).

Thus, it is concluded that, in order to ensure a good skin penetration, the mix ratio of 1:4 is desirable for fabricating the second device 800. The success penetration rate of the micro-needles 804 with the maltose sharp tips 806b is lower than that of the micro-needles 804 with SU-8 sharp tips 806a when the test results of buckling force show an inverse trend. Although the micro-needles 804 with the maltose sharp tips 806b are able to withstand a higher buckling force, a higher force also needs to be applied to the micro-needles in order to penetrate the skin surface. This is because the thicker needle body of maltose sharp tip 806b affects a wider surface area during skin penetration, thus resulting in a higher possibility of being bent during skin penetration. 11(d)(i-1) and (11(d)(i-2) in FIG. 11C shows the histology image of skin penetration by a micro-needle 804 with the SU-8 sharp tip 806a and the maltose sharp tip 806b. Specifically, a micro-channel created by the maltose sharp tip 806b (see 11(d)(i-2)) is much broader than that a micro-channel made by the SU-8 sharp tip 806a (see 11(d)(i-1)), which confirms that the maltose sharp tip 806b requires more force to penetrate the skin surface.

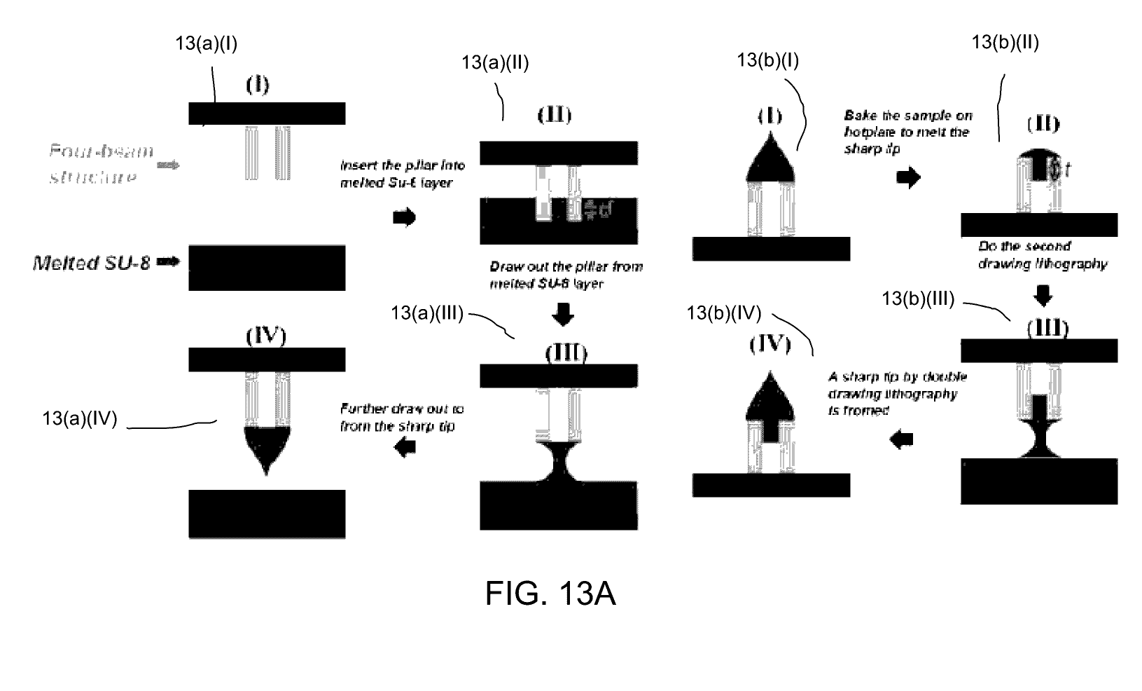

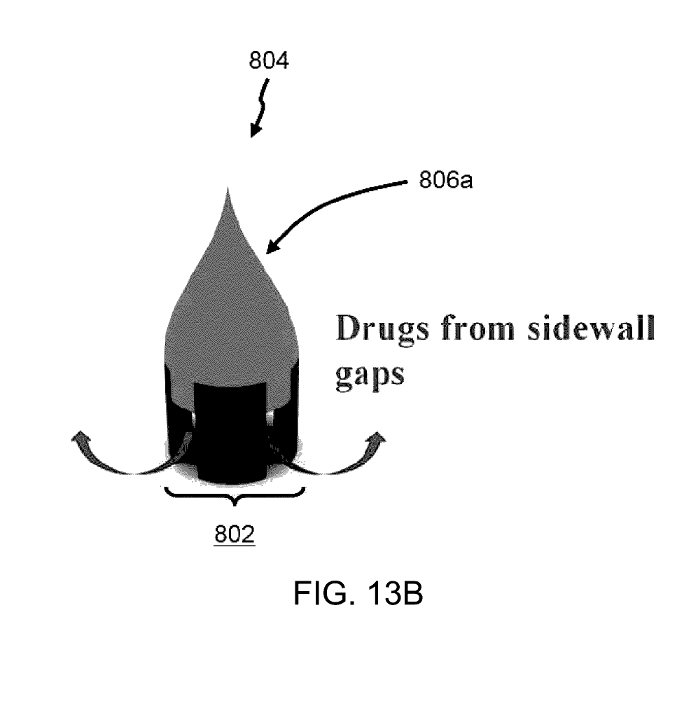

Another parameter that affects the buckling force of the bendable micro-needles 804 is the angular of the PDMS pillars 802 arranged beneath the sharp tips 806a, 806b. When the angular of the PDMS pillars 802 decreases from 60° to 30° and the angular of gaps between the PDMS pillars 802 increases from 30° to 60°, the anchor of the rigid sharp tip 806a, 806b takes a higher ratio, making the micro-needle 804 more rigid and enhancing the buckling force. However, for manufacturing the SU-8 sharp tips 806a by using double drawing lithography as shown in FIGS. 13A-13B (to be elaborated below), the baking time to melt the SU-8 sharp tips 806a assembled by the first step drawing cannot be well controlled to realize a partially filled gap for the samples with pillar angular lower than 55°. Because of the large gap angular, once melted, the SU-8 always fills the whole gap, leaving no outlet for the drug to be delivered. Therefore, for the micro-needles 804 with the SU-8 sharp tips 806a, only the pillars of 60° pillar angular are used. The drawing process to assemble the maltose sharp tips 806b is not limited by the pillar angular, thus the buckling force of micro-needles 804 with the maltose tips 806b by changing the pillar angular is evaluated and reported in FIG. 11E. For each pillar angular, nine micro-needles 804 were tested. When the pillar angular decreases from 60° to 30° (FIG. 11D), the buckling force of the individual micro-needle 804 increases from 0.43 N to 0.92 N because the entire micro-needle 804 becomes more rigid. However, the contact area between maltose and PDMS pillars 802 also decreases with the decrease of the pillar angular. Thus the maltose sharp tips 806b cannot be well-fixed within the PDMS pillars 802 and tends to break or detach from the PDMS pillars 802 when the vertical force is applied to make the micro-needles 804 bend. For the case of 30° pillar angular, eight out of nine micro-needles 804 showed breakage of the sharp tips 806a, 806b. The possibility of the needle breakage decreases with the increase of pillar angular. For the needles of pillar angular more than 50°, no needle breakage was observed in the buckling force tests. As a conclusion, to avoid needle breakage and have buckling force as high as possible, a pillar angular of 50° is the optimum value for micro-needles 804 having the maltose sharp tips 806b.

### 4.2 Using Double Drawing Lithography to Manufacture the Micro-Needles

Referring to FIGS. 13A-13B, which show usage of double drawing lithography to form the SU-8 sharp tips 806a onto the flexible substrate to manufacture the second device 800, a pre-baked SU-8 layer is first prepared on a Si substrate. Then the flexible substrate is mounted above the SU-8 layer, and the SU-8 layer is baked to make melt the SU-8 layer as shown in 13(a)(I). Then a portion of the protrusions 802 (i.e. the four-beam-pillar bases) of the flexible substrate are inserted into the melted SU-8 layer to a depth d, as shown in 13(a)(II). Next, the protrusions 802 are slowly drawn out from the melted SU-8 layer. During the drawing out process, some of the melted SU-8 attach onto the portion of the respective protrusions 802 that was dipped into the melted SU-8 layer, and so a SU-8 bridge is formed between the melted SU-8 layer and each protrusion 802 as shown in 13(a)(III). The drawing out of the protrusions 802 is continued until the respective SU-8 bridges are broken to form respective SU-8 sharp tips 806a, as shown in 13(a)(IV). It is to be appreciated that at this stage, the SU-8 sharp tips 806a formed are not in the final form, and are hollow too.

Then, the entire flexible substrate with the newly formed SU-8 sharp tips 806a are baked in an oven at 120° C. to melt the hollowed SU-8 sharp tips 806a as shown in 13(b)(II). The molten SU-8 reflows into the gaps between the respective protrusions 802 and the molten sharp tips 806a are now transformed into a dome shape. Then a second drawing process is performed on the top of molten dome-shaped SU-8 portions arranged on the protrusions 802 to form sharp and solid tips 806a, as shown in 13(b)(III) and 13(b)(IV). The flowing depth t of the melted SU-8 in the gaps is controlled by changing the baking time disclosed in 13(b)(II). As each protrusion 802 used in the drawing lithography is made in the form of a four-beam structure, which means there are gaps along the sidewalls, fluid drug medications thus are able to flow out from the respective micro-needles 804 from the gaps along the sidewalls as shown in FIG. 13B.

### 5. Summary

The bendable micro-needles 804 of the second device 800 are configured to tolerate the deformation associated with skin stretching (due to relative movement) without breakage, when the skin patch 802 is applied onto the joint areas of a user, such as the elbow and knuckle for osteoporosis treatment. In other cases, such as treatment for diabetes, in which conventional micro-needle patches are normally applied on the arm or abdomen, a lateral movement between the conventional micro-needle patches and skin surface may occur due to the occasional touch or friction. In such cases, by using the proposed second device 800, the bendable micro-needles 804 will be dragged out of the skin instead of leaving a broken needle in the skin when lateral movement occurs, as opposed to using conventional micro-needle patches. The sharp tips 806a, 806b assembled onto the reversibly deformable protrusions 802 of the flexible substrate may either be non-bio-dissolvable, i.e. made of SU-8, or bio-dissolvable, i.e. made of maltose. For the configuration with the SU-8 sharp tips 806a, the recesses 803 of the micro-needles 804, fluid connecting to the drug reservoirs 110, are always exposed to air. Accordingly, both water-soluble and lipophilic drugs may immediately be delivered just after skin penetration. For the configuration with the maltose sharp tips 806b, the recesses 803 may be fully encapsulated by maltose to inhibit the solvent evaporation of lipophilic drug formulation. Hence, the drug can be delivered when the maltose sharp tips 806b have melted after skin penetration. But the micro-needles 804 with the maltose sharp tips 806b are however not suitable for administering water-soluble drug formulations, because the evaporation of water in the formulations may cause melting of the maltose sharp tips 806b. Therefore, the water-soluble drug formulations may only be stored in the proposed skin patches 802 configured with the SU-8 sharp tips 806a.

Further, acrylic medical bandages are conventionally widely used for medical patches. However, there are increasing demands on less-irritating, biocompatible medical bondages, as aging skins (in older patients) are more sensitive and vulnerable to a prolonged exposure, e.g. insulin delivery, as in the case of conventional medical patches. Dry adhesives, which are inspired by the hierarchical structure on Gecko foot hair, possess several advantages compared with conventional acrylic medical bandages: Firstly, dry adhesive shows repeatable and restorable adhesion with surface cleaning after each usage. Secondly, the physical structure to generate adhesive force is less affected by surface contamination, oxidation and other environmental stimuli. Thirdly, the recesses 803 of the respective protrusions 802 for ventilation of air should provide better overall bio-compatibility. Hence, the dry adhesives are adopted by the proposed skin patch 802 (as detailed in Section 2.3) in order to better adhere the skin patch 802 onto the skin of users.

### 6. Characterization of the Proposed Skin Patch for Insulin Delivery Test

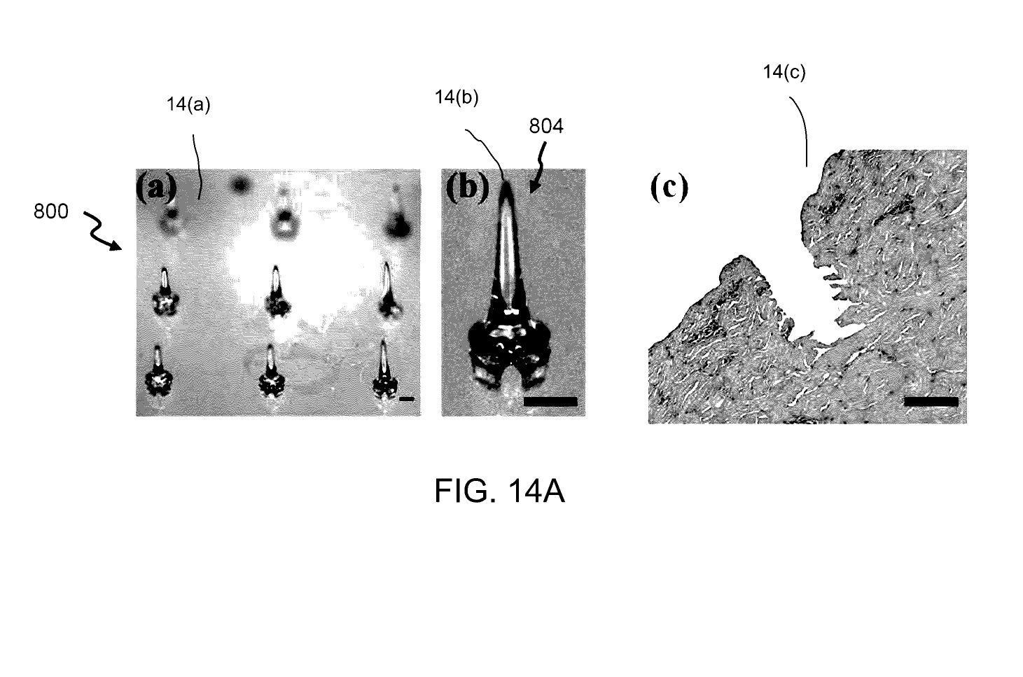

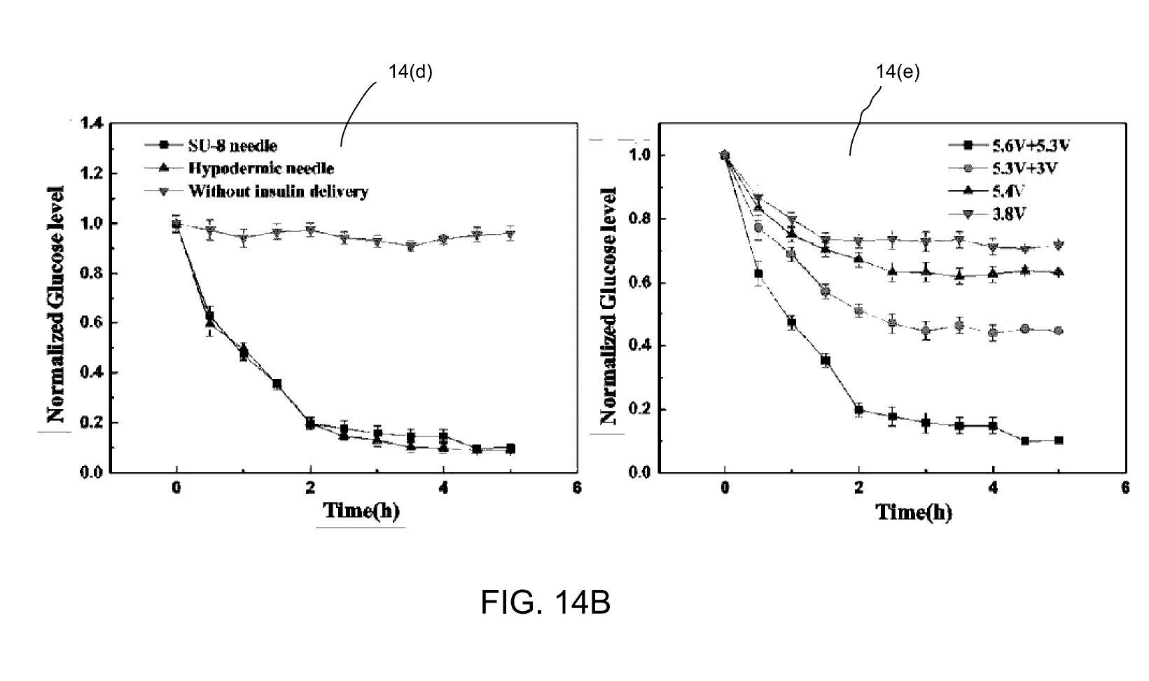

To evaluate the disclosed skin patch 802 and also to confirm that the first device 100 has ideal features for efficient drug volume control, evaluation tests on the skin patch 802 for transdermal delivery of insulin was performed in vivo. As a powerful approach for various biomedical researches such as transdermal drug delivery and transdermal bio-sensing, the skin patch 802 is evaluated for skin penetration and insulin delivery performance. Penetration tests on mouse cadaver skin were conducted to characterize the penetration capability of the micro-needles 804 configured with the SU-8 sharp tips 806a. A histology image of the skin at the site of one micro-needle 804 penetration confirms that the associated SU-8 sharp tip 806a was not broken during the insertion steps, as shown as 14(c) in FIG. 14A.

It is to be appreciated that all procedures for the evaluations were performed under protocol and approved by the Institutional Animal Care and Use Committee at the National University of Singapore (NUS). Particularly, Sprague-Dawley rats with an average weight of 200-250 g were injected with 50 mg kg⁻¹ of streptozotocine (Sigma-Aldrich, Singapore) in citrate buffer (pH 4.2) via intraperitoneal injection to generate a diabetic animal model. The rats were kept with free access to food and water for 3 days. Then the rats' blood glucose levels were checked by a glucometer (Accu-Chek, USA). Following on, rats with blood glucose levels determined to be between 16 and 30 mM were selected, and body hairs on the abdomen skin of the selected rats were removed by a razor 24 hours before the experiment. All these rats were divided into 3 groups (i.e. Group 1, Group 2, and Group 3) and each group contained 3 subject rats.

The evaluations without liquid volume control were first conducted to confirm the drug delivery capability of the skin patch 802. Group 1 was devised as a negative control group, in which the blood glucose level was only tested throughout the duration of the tests. Group 2 was then framed as an experimental group. After the rats were anesthetized, a skin patch 802 with insulin loaded was applied onto the abdomen skin surface. The pump 104 was pressed to deliver all the insulin (i.e. 10 IUmL⁻¹) contained in the drug reservoirs 110. The totally volume of all the drug reservoirs 110 is about 246 μL. In Group 3, after the rats were anesthetized, 10 IUmL⁻¹ of Lispro insulin was injected subcutaneously with a 29G hypodermic needle into the rats (2.5 IUkg⁻¹) as a positive control experiment.

Blood samples were taken from the tail vein of each rat for all the groups, at every 30 minutes interval, after the evaluations have begun. The blood glucose level monitoring lasted for 5.5 hours. A glucometer (Accu-Chek, USA) was used to measure the corresponding blood glucose levels. The results are shown in 14(d) of FIG. 14B. Specifically, the blood glucose levels in rats treated with the proposed skin patch 802 dropped continuously during the 5.5 hours insulin delivery period and was quite stable after three hours. It was significantly different compared with the negative control group (i.e. Group 1), where insulin solution was not administered to the rats. Remarkably, the change of the blood glucose level in the positive control experiments in both groups, i.e. using the skin patch 802 and a hypodermic needle, was found to be about the same. This experiment successfully proved the feasibility of using proposed skin patch 802 to deliver macromolecules like insulin.

A detailed study was also conducted in order to study the ability of manual control function for insulin delivery, which is supported by the microfluidic device 100 and the sensor 102. During testing with the skin patch 802, the delivery volume was controlled by adjusting the pressing force on the pump 104. The output voltage of the sensor 102 was recorded to confirm the different volume delivered during the tests, as shown in 14(e). There were 4 groups in this current study. For the first 2 groups, only one time pressing was applied during the test. The output voltages of the sensor 102 of the respective groups were measured to be 3.8 V and 5.4 V. For the last 2 groups, the pump 104 was pressed twice and the output voltages of the sensor 102 were measured as 5.3 V+3 V and 5.6 V+5.3 V, respectively. The change of the blood glucose level is shown in 14(e). For the group with a higher voltage output, which correspondingly implies a larger volume of insulin delivery, the blood glucose level drops more. But for all the groups, the blood glucose level stabilized at a certain level after three hours. The experiment confirms that the manual control delivery mechanism with the sensor 102 is able to successfully monitor insulin delivery and further control the blood glucose level. However, during the insulin delivery, the micro-needles 804 were immersed within the skin, blocking the outlets of the micro-needles 804. Thus, the flow resistance of the microfluidic channels in the microfluidic device 100 accordingly increased significantly, which resulted in a slight deviation of the actual delivery volume from the in-vitro calibration. From the evaluations, a general observation is that the actual delivery volume of in-vivo was around 10% lower than that of in-vitro calibration in the situation of drug delivery when the micro-needles 804 were embedded in the skin.

While the invention has been illustrated and described in detail in the drawings and foregoing description, such illustration and description are to be considered illustrative or exemplary, and not restrictive; the invention is not limited to the disclosed embodiments. Other variations to the disclosed embodiments can be understood and effected by those skilled in the art in practising the claimed invention. For example, the micro-features may be configured with other suitable shapes, and not necessarily a pyramid shape. Then, the relative positioning of the first and second portions 112, 114 of the sensor 102 may be interchanged, i.e. the first portion 112 is arranged to abut the pump 104, whereas the second portion 114 is then arranged in direct opposition to the first portion 112. In addition, the fluid/drug reservoirs 110 may also be arranged before or after the second check valve 106b, and need not always be in a series arrangement with the second check valve 106b.

1. A microfluidic device comprising:
a triboelectric sensor;
an elastically deformable pump arranged to transfer fluid to at least one fluid outlet and triboelectrically activate the sensor; and
first and second check valves respectively arranged at the inlet and outlet of the pump to control fluid transfer in and out of the pump,
wherein when the pump is actuated in conjunction with the check valves to transfer a volume of fluid, at least a portion of the sensor is triboelectrically activated according to an amount of deformation of the pump to generate a corresponding output voltage signal for enabling the volume of fluid transferred by the deformed pump to be determined.

2. The device of claim 1, wherein the pump includes being arranged to abut the sensor.

3. The device of claim 1, further comprising at least one fluid reservoir for holding a fluid drug medication, which is to be delivered together with the volume of fluid.

4. The device of claim 3, wherein the fluid drug medication includes insulin.

5. The device of claim 1, wherein the pump includes being formed of polydimethylsiloxane (PDMS).

6. The device of claim 1, further including a plurality of triboelectric energy harvesters configured in a stacked arrangement for generating a collective output voltage signal.

7. The device of claim 6, further comprising a convertor circuit, and at least one power component, wherein the convertor circuit is configured to convert the collective output voltage signal into electricity for powering the power component.

8. The device of claim 7, wherein the power component includes an integrated circuit, a microprocessor, and a LCD reading panel.

9. The device of claim 1, wherein the sensor includes first and second portions in opposing arrangement and separated by an air gap, the first portion having at least a first dielectric layer, and the second portion having at least a second dielectric layer coated with a layer of parylene.

10. The device of claim 9, wherein the first and second dielectric layers are formed of polydimethylsiloxane (PDMS).

11. The device of claim 9, wherein a surface of the first electric layer is further coated with a metal layer, in which the surface is in opposition to the second portion.

12. The device of claim 11, wherein the metal layer includes an aluminium layer.

13. The device of claim 9, wherein a surface of the second electric layer is further regularly arranged with a plurality of micro-features, in which the surface is in opposition to the first portion.

14. The device of claim 13, wherein each micro-feature has a pyramid shape.

15. The device of claim 9, wherein the second portion is arranged adjacent to the pump.

16. The device of claim 1, further comprising a micro-needle array device which includes:
a flexible substrate formed with a plurality of base protrusions which are elastically deformable, each protrusion having a plurality of recesses for enabling fluid transfer; and
a plurality of micro-needles co-axially arranged on the respective protrusions, the micro-needles being substantially rigid,
wherein when a lateral force is applied onto the device, the micro-needles are displaced off-axis relative to the protrusions due to deformation of the protrusions, and when the lateral force is removed, the micro-needles return to the co-axial arrangement; and
wherein the device is incorporated with the micro-needle array and a plurality of dry adhesive patches to form a skin patch adapted for transdermal drug delivery.

17. The device of claim 16, wherein the flexible substrate is formed of polydimethylsiloxane (PDMS), and the micro-needles are formed of SU-8 photoresist or maltose.

18. A method of using a microfluidic device, which includes a triboelectric sensor; an elastically deformable pump arranged to transfer fluid to at least one fluid outlet and triboelectrically activate the sensor; and first and second check valves respectively arranged at the inlet and outlet of the pump to control fluid transfer in and out of the pump, the method comprises:
actuating the pump in conjunction with the check valves to transfer a volume of fluid, and to cause at least a portion of the sensor to be triboelectrically activated according to an amount of deformation of the pump; and
generating a corresponding output voltage signal by the activated sensor for enabling the volume of fluid transferred by the deformed pump to be determined.

19. A micro-needle array device comprising:
a flexible substrate formed with a plurality of base protrusions which are elastically deformable, each protrusion having a plurality of recesses for enabling fluid transfer; and
a plurality of micro-needles co-axially arranged on the respective protrusions, the micro-needles being substantially rigid,
wherein when a lateral force is applied to the device, the micro-needles are displaced off-axis relative to the protrusions due to elastic deformation of the protrusions caused by the force, and when the force is removed, the micro-needles return to the co-axial arrangement.

20. The device of claim 19, wherein the flexible substrate is formed of polydimethylsiloxane (PDMS), and the micro-needles are formed of SU-8 photoresist or maltose.
